(19) United States
(12) Patent Application Publication
(10) Pub. No.: US 2004/0116417 A1
(43) Pub. Date: Jun. 17, 2004

(54) Thiohydantoins and use thereof for treating diabetes

(76) Inventors: Benaissa Boubia, Saint Apollinaire (FR); Evelyne Chaput, Dijon (FR); Khan Ou, Hauteville-les-Dijon (FR); Philippe Ratel, Ahuy (FR)

Correspondence Address: MERCHANT & GOULD PC, P.O. BOX 2903, MINNEAPOLIS, MN, 55402-0903, US

(21) Appl. No.: 10/473,032

(22) PCT Filed: Apr. 4, 2002
(86) PCT No.: PCT/FR02/01167

(30) Foreign Application Priority Data
Apr. 4, 2001 (FR) 0104552

(51) Int. Cl. A61K 31/541; A61K 31/5377; A61K 31/496; A61K 31/454; A61K 31/4164; C 07D 4 3/02; C07D 413/02; C07D 417/02

(52) U.S. Cl. 514/227.8; 514/235.5; 514/254.05; 514/326; 514/389; 544/60; 544/139; 544/370; 546/210; 548/301.4; 548/317.1

(57) ABSTRACT

The invention relates to 2-thiohydantoin derivative compounds selected from compounds of general formula (I):

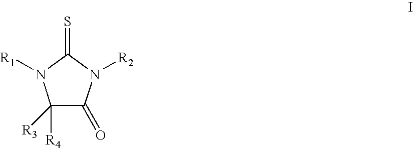

as defined in the claims, and to their addition salts with an acid, notably pharmaceutically acceptable salts.

The invention also relates to their method of preparation, the pharmaceutical compositions containing them, and their use as pharmacologically active substance, notably in the case of the treatment of diabetes and diseases caused by a hyperglycaemia, hypertriglyceridaemiae, dyslipidaemiae or obesity.

The present invention relates to novel thiohydantoin (or 2-thioxo-4-imidazolidinone) derivative compounds, their method of preparation and their use as active principles for the preparation of medicaments which are notably intended for treating diabetes.

### PRIOR ART

The chemistry of thiohydantoin-type compounds is known for many years. Certain derivatives of this heterocycle have been used in the field of photography, as described for example in U.S. Pat. No. 2,551,134 or JP 81 111847, or in the field of pesticides, essentially herbicides or fungicides, as described for example in U.S. Pat. No. 3,798,233, in the publications Indian J. Chem.; 1982, Vol 21B, p. 162-164, J. Indian Chem. Soc., Vol 58(10), p. 994-995, Chem. Abst. 67. 82381v, Indian J. Chem., 1979, vol 18B, p. 257-261, and U.S. Pat. No. 4,473,393. More recently, compounds containing a thiohydantoin ring have been prepared with the view to obtaining products which are active in therapeutics. For example, U.S. Pat. No. 3,923,994 describes the use of 3-aryl-2-thiohydantoins for their anti-arthritic activity. U.S. Pat. No. 3,984,430 proposes novel thiohydantoins for treating ulcers. Indian J. Chem. (1978), Vol 16B, p 71-72, describes coumaryl-thiohydantoins which are active against tuberculosis. U.S. Pat. No. 4,312,881 claims acids and esters which comprise a 2-thiohydantoin ring and which have prostaglandin-type activity. Chem. Pharm. Bull., (1982), Vol 30, no 9, p. 3244-3254, describes the inhibition of aldose-reductases by compounds of 1-(phenylsulphonyl)-2-thiohydantoin type. Il Farmaco, Ed Scientifico (1983), Vol 38, no 6, p. 383-390, proposes 3-dialkylaminopropyl-2-thiohydantoins as anti-arrhythmic agents. WO 96/04248 A describes amide- or sulphonamide-type derivatives of 2-thiohydantoin which are antagonists of angiotensin II. WO 97/19932 A claims the use of 2-thiohydantoin derivatives for increasing HDL content. WO 98/33776 cites a bank of compounds which are obtained by combinatorial chemistry and which are tested for their anti-microbial or analgesic properties. Finally, WO 93/18057 and EP 584694 describe acids or esters which comprise a 2-thiohydantoin ring and which are platelet aggregation inhibitors.

Preparations of compounds comprising a 2-thiohydantoin ring without indication of the industrial usefulness have also been described for example in J. Prakt. Chem., Vol. 333(2), p. 261-266, Indian J. Chem., (1974), vol 12, no 6, p. 577-579, Chem. Abstr., 68 (1968), 87240d, and Organic Magn. Resonance, vol 19, (1) p. 27-30.

### AIM OF THE INVENTION

The present invention relates to novel compounds comprising the heterocycle 2-thiohydantoin (or 2-thioxo-4-imidazolidinone) in their structure, as well as to their method of preparation and their use in therapeutics, notably for the preparation of a medicament intended for treating diabetes, diseases caused by a hyperglycaemia, hypertriglyceridaemiae, dyslipidaemiae, or obesity.

### DESCRIPTION

According to the invention, novel 2-thiohydantoin derivatives are proposed which are selected from:

a) compounds of formula

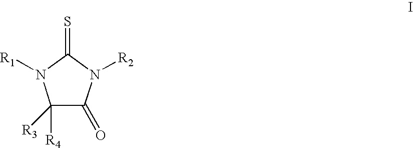

in which

R₁ represents an aromatic ring which is non-substituted or substituted with one or more atoms or groups of atoms selected from halogens, linear or branched C₁-C₄ alkoxy, linear, branched or cyclic C₁-C₄ alkyl, linear or branched C₁-C₄ alkylthio, nitro, trifluoromethyl, trifluoromethoxy, methylenedioxy, or

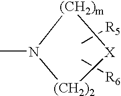

groups,

R₂ represents:

a hydrogen atom,

a linear, branched or cyclic C₁-C₇ alkyl group, optionally having one or more oxygen atoms,

a C₁-C₃ haloalkyl group,

a linear or branched C₃-C₅ alkenyl group,

a linear or branched C₃-C₄ alkynyl group,

a C₂-C₆ hydroxyalkyl group,

a C₂-C₄ aminoalkyl group,

a C₂-C₃ cyanoalkyl group,

a linear or branched C₁-C₃ alkyl group, which is substituted with one or more R₇ substituents, or

an aromatic ring which is non-substituted or substituted with one or more atoms or groups of atoms selected from halogens, linear or branched C₁-C₄ alkoxy, linear, branched or cyclic C₁-C₄ alkyl, linear or branched C₁-C₄ alkylthio, amino, cyano, hydroxy, nitro, trifluoromethyl, trifluoromethoxy, methylenedioxy, ethylenedioxy, difluoromethylenedioxy, aminosulphonyl, dimethylamino, C₁-C₃ hydroxyalkyl, carboxylic acid, C₂-C₃ alkyl ester, methanesulphonylamino, benzenesulphonylamino, t-butoxycarbonylamino, or

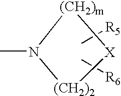

groups,

R₃, R₅ and R₆ each independently represent a hydrogen atom or a C₁-C₄ alkyl group,

R₄ represents a hydrogen atom, a C₁-C₄ alkyl group or a hydroxy group, or,

R₃ and R₄ together form a methylene group, or

R₅ and R₆ together form an ethylene group —CH₂—CH₂—,

R₇ represents a carboxylic acid group which is free or esterified with a C₁-C₃ alkyl group, a phenyl ring which is non-substituted or substituted with one or more methoxy, phenyl or methylenedioxy groups, a 2-furyl ring, a 2-, 3- or 4-pyridinyl ring or a 4-morpholinyl group,

m=2 or 3,

X represents an oxygen atom, a sulphur atom, a sulphoxide group, a sulphonyl group, a carbonyl group, a

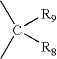

group, or a:

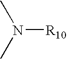

group,

R₈ represents a hydrogen atom, a hydroxy group, a C₁-C₂ hydroxyalkyl group, a benzoyl group or a CO₂CH₃ group,

R₉ represents a hydrogen atom or forms, with R₈, an ethylenedioxy group, and

R₁₀ represents a methyl group, a C₂-C₄ hydroxyalkyl group, a 1-oxo-C₂-C₄-alkyl group, an SO₂N(CH₃)₂ group, a 2-pyridinyl group or a 2-pyrimidinyl group,

on the condition that at least one of the R₁ and R₂ substituents represents an aromatic ring which is substituted at least with one

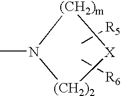

group,

and

b) addition salts of the compounds of formula I with an acid, notably pharmaceutically acceptable salts.

The invention also comprises, when the R₃ and R₄ substituents are different, compounds of R-configuration, compounds of S-configuration, and their mixtures.

The invention also relates to compounds of formula I or their addition salts with an acid, which are pharmaceutically acceptable, for their use as a pharmacologically active substance.

The invention relates in particular to the use of at least one compound according to formula I above as an active principle for the preparation of a medicament intended for a use in therapeutics, notably for fighting against diseases caused by a hyperglycaemia, diabetes, hypertriglyceridaemiae, dyslipidaemiae or obesity.

### DETAILED DESCRIPTION

In formula I, which represents the compounds of the invention:

A <<C₁-C₄ alkyl group >> is understood to be a linear, branched or even cyclic, saturated hydrocarbon chain having 1 to 4 carbon atoms. Examples of C₁-C₄ alkyl groups include methyl, ethyl, propyl, butyl, 1-methylethyl, cyclopropyl, 1-methylpropyl, 2-methylpropyl, or 1,1-dimethylethyl groups. A << C₁-C₇ alkyl group optionally having one or more oxygen atoms >> is understood to be a linear, branched or ring-containing, saturated hydrocarbon chain having 1 to 7 carbon atoms, it being possible for said chain to include one or more non-consecutive oxygen atoms between 2 carbon atoms. Examples of C₁-C₇ alkyl groups optionally having one or more oxygen atoms include the groups which are cited above as well as, notably, pentyl, hexyl, heptyl, 1-methylethyl, cyclohexyl, cyclohexylmethyl, methylcyclohexyl, methoxyethyl, ethoxyethyl, ethoxyethoxyethyl or even tetrahydropyranyloxyalkyl groups.

When a phenyl group is substituted, the substituent can be located in the ortho, meta or para position, the para position being preferred.

A << C₁-C₃ haloalkyl group >> is understood to mean a C₁-C₃ alkyl group which bears at least one halogen atom selected from fluorine, chlorine or bromine, preferably fluorine, for example a trifluoromethyl or 2,2,2-trifluoroethyl group.

A <<linear or branched C₁-C₄ alkoxy group>> is understood to mean methoxy, ethoxy, propoxy, butoxy or 1-methylethoxy groups.

A <<C₃-C₅ alkenyl group>> is understood to mean a linear or branched chain which has in its structure a double bond between 2 carbons.

A <<C₃-C₄ alkynyl group>> is understood to mean a linear or branched chain which has in its structure a triple bond between 2 carbons.

A <<C₂-C₆ hydroxyalkyl group>> is understood to mean an alkyl group having 2 to 6 carbon atoms which is substituted with a hydroxy group. Examples of a C₂-C₆ hydroxyalkyl group include 2-hydroxyethyl, 2-hydroxypropyl, 3-hydroxypropyl, 4-hydroxybutyl, 5-hydroxypentyl, or 6-hydroxyhexyl groups.

A <<C₂-C₄ aminoalkyl group>> is understood to mean an alkyl group having 2 to 4 carbon atoms, which is substituted with an amino group NH₂, it being possible for said amino group to be protected by a group of atoms known to the person skilled in the art, e.g. an alkylsulphonyl group or a t-butoxycarbonyl (Boc) group.

A <<C₂-C₃ cyanoalkyl group>> is understood to mean an alkyl group having one or two carbon atoms, which is substituted with a cyano group.

Examples of an aromatic ring are phenyl, 2- or 3-thienyl, 2- or 3-furyl 2-, 3- or 4-pyridinyl, 1- or 2-naphthyl, indolyl, 1-H-imidazolyl, 1-H-benzimidazolyl, benzotriazolyl, 1,3-dihydro-2-oxo-benzimidazolyl, 1,3-dihydro-2-oxo-indolyl, 2H-2-oxo-benzopyranyl, 2H-4H-3-oxo-1,4-benzoxazinyl rings.

A <<halogen>> is understood to mean fluorine, chlorine or bromine, the preferred halogen atoms in compounds of formula I according to the invention being fluorine and chlorine.

Compounds of formula I which bear an amine function by the presence of a nitrogen-containing heterocycle or by the presence of an amine substituent, can be salified by a reaction with an acid which is non-toxic and which is acceptable in therapeutics. Mineral acids such as hydrochloric, hydrobromic, phosphoric and sulphuric acids, or organic acids such as methanesulphonic, benzenesulphonic, citric, maleic, fumaric, oxalic, lactic, tartaric or trifluoroacetic acids, can be selected from these acids.

A preferred family of the compounds of formula (I) of the invention includes:

a) compounds of formula

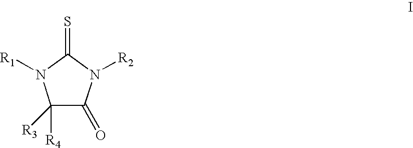

in which

R₁ represents a phenyl ring which is optionally substituted with one or more atoms or groups of atoms selected from halogens, linear C₁-C₄ alkyl or

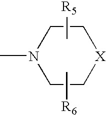

groups,

R₂ represents

a linear, branched or cyclic C₁-C₇ alkyl group,

a linear C₃-C₅ alkenyl group, or

a phenyl, 2-thienyl or 3-pyridinyl ring, which is optionally substituted with one or more atoms or groups of atoms selected from halogens, linear or branched C₁-C₄ alkoxy, linear C₁-C₄ alkyl, linear C₁-C₄ alkylthio, amino, hydroxy, nitro, trifluoromethyl, trifluoromethoxy, methylenedioxy or

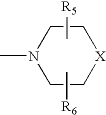

groups,

R₄ represents a hydrogen atom, a linear C₁-C₄ alkyl group, or a hydroxy group,

R₃, R₅, and R₆ each independently represent a hydrogen atom or a linear C₁-C₄ alkyl group,

X represents an oxygen atom, a sulphoxide group or a carbon atom which is substituted with a C₁-C₂ hydroxyalkyl group,

on the condition that at least one of the R₁ and R₂ substituents represents an aromatic ring which is substituted at least with a

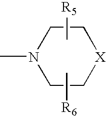

group,

and

b) addition salts of compounds of formula I with an acid, notably pharmaceutically acceptable salts.

Compounds of formula I in which R₁ represents a phenyl group which is substituted at least in the para position with a

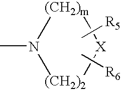

group, are more particularly preferred amongst the compounds of the invention, and from these compounds, those in which X represents an oxygen atom, m=2 and R₅ and R₆ each represent a hydrogen atom or a methyl group.

Compounds of formula I in which R₃ represents a hydrogen atom and R₄ represents a methyl group are also preferred.

Compounds of formula I can be prepared according to a first general method A which consists in:

1) allowing an amino acid of formula:

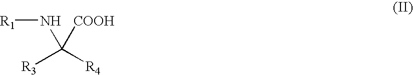

in which

R₁ represents an aromatic ring which is non-substituted or substituted with one or more atoms or groups of atoms selected from halogens, linear or branched C₁-C₄ alkoxy, linear, branched or cyclic C₁-C₄ alkyl, linear or branched C₁-C₄ alkylthio, nitro, trifluoromethyl, trifluoromethoxy, methylenedioxy or

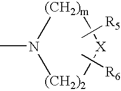

groups,

m represents 2 or 3,

X represents an oxygen atom, a sulphur atom, a sulphoxide group, a sulphonyl group, a carbonyl group, a

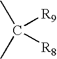

group, or a:

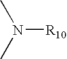

group,

R₃, R₄, R₅ and R₆ each independently represent a hydrogen atom or a C₁-C₄ alkyl group,

R₈ represents a hydrogen atom, a hydroxy group, a C₁-C₂ hydroxyalkyl group, a benzoyl group or a CO₂CH₃ group,

R₉ represents a hydrogen atom or forms, with R₈, an ethylenedioxy group,

R₁₀ represents a methyl group, a C₂-C₄ hydroxyalkyl group, a 1-oxo-C₂-C₄-alkyl group, an SO₂N(CH₃)₂ group, a 2-pyridinyl group or a 2-pyrimidinyl group,

to react with an isothiocyanate of formula

R₂—N═C═S (III)

in which R₂ represents

a linear, branched or cyclic C₁-C₇ alkyl group, optionally having one or more oxygen atoms,

a C₁-C₃ haloalkyl group,

a linear or branched C₃-C₅ alkenyl group,

a linear or branched C₃-C₄ alkynyl group,

a C₂-C₆ hydroxyalkyl group,

a protected C₂-C₄ aminoalkyl group,

a C₂-C₃ cyanoalkyl group,

a linear or branched C₁-C₃ alkyl group, which is optionally substituted with one or more R₇ substituents, or

an aromatic ring which is non-substituted or substituted with one or more atoms or groups of atoms selected from halogens, linear or branched C₁-C₄ alkoxy, linear, branched or cyclic C₁-C₄ alkyl, linear or branched C₁-C₄ alkylthio, cyano, hydroxy, nitro, trifluoromethyl, trifluoromethoxy, methylenedioxy, ethylenedioxy, difluoromethylenedioxy, aminosulphonyl, dimethylamino, C₁-C₃ hydroxyalkyl, carboxylic acid, C₂-C₃ alkyl ester, methanesulphonylamino, benzenesulphonylamino, t-butoxycarbonylamino, or

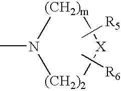

groups,

in a solvent, such as acetonitrile or dichloromethane for example, in the presence of an aprotic base, notably such as triethylamine, at a temperature of between 10° C. and the reflux temperature of the solvent, for 2 to 4 hours, to obtain the compound of formula I

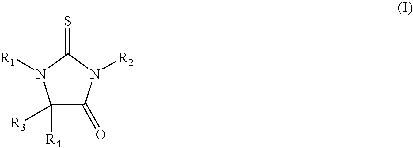

in which R₁, R₂, R₃, R₄ keep the same meaning as above, it being understood that at least one of the R₁ and R₂ groups contains in its structure an aromatic ring which is substituted at least by the

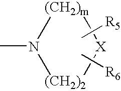

group, as defined above;

and,

2) if necessary, obtaining the addition salt of the compound of formula I above with an organic or mineral acid.

According to a second method E of preparation of a compound according to the invention, the following steps are carried out, which consist in:

1) allowing an amino acid ester of formula (IIa)

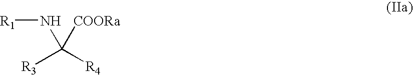

in which R₁, R₃ and R₄ have a meaning which is analogous to that of the R₁, R₃ and R₄ substituents which are noted for the compound of formula II which is described in the method A, and Ra represents a C₁-C₃ alkyl group, preferably an ethyl group,

to react with an isothiocyanate of formula

R₂—N═C═S (III)

as described above for the method A,

in a solvent, such as toluene for example, and in the presence of a weak acid, such as acetic acid, at a temperature of between 50° C. and the boiling temperature of the solvent, for 2 to 25 hours, to obtain the compound of formula I

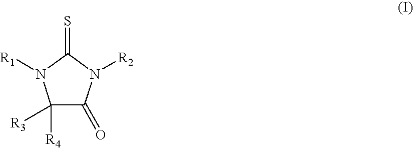

in which R₁, R₂, R₃, R₄ keep the same meaning as above, it being understood that at least one of the R₁ and R₂ groups contains in its structure an aromatic ring which is substituted at least by the

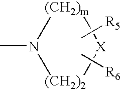

group, as defined above;

and,

2) if necessary, obtaining the addition salt of the compound of formula I above with an organic or mineral acid.

In a variant of step 1) of the method E described above, it is possible for the compounds of formula IIa to be allowed to react according to a method F, which consists in mixing the 2 compounds IIa and III well, without solvent, and in keeping the mixture at a, temperature of about 110 to 130° C., for 0.5 to 3 hours, to obtain the compound of formula I in which R₁, R₂, R₃ and R₄ keep the same meaning as in the starting materials.

According to a second variant M of step 1) of the method E described above, it is possible for the compounds of formula IIa and III to be allowed to react according to a method consisting in mixing the compounds IIa and III well in a tube or a PTFE reactor in the presence of a small amount of acetic acid and heating the mixture for 1 to 15 minutes by means of a microwave radiation, to obtain the compound of formula I in which R₁, R₂, R₃ and R₄ keep the same meaning as in the starting materials.

It is possible for compounds of formula II to be obtained by reaction of an amine of formula

R₁—NH₂ (IV)

in which R₁ represents the same meaning as above, with a halogen-containing acid of formula

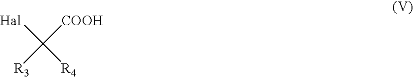

in which Hal represents a halogen, preferably bromine, R₃ and R₄ each independently represent a hydrogen atom or a C₁-C₄ alkyl group,

preferably in the absence of solvent and in the presence of sodium bicarbonate, at a temperature of between 60 and 140° C., for 0.5 to 10 hours, in order to obtain the acid of formula

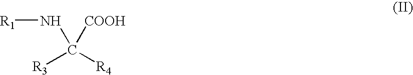

in which R₁, R₃ and R₄ keep the same meaning as in the starting materials,

It is possible for compounds of formula IIa to be obtained by reaction of an amine of formula

R₁—NH₂ (IV)

in which R₁ represents the same meaning as above, with an α-halogenated ester of formula

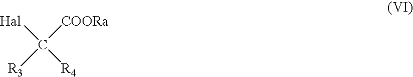

in which Hal represents a halogen, preferably bromine, Ra represents a C₁-C₃ alkyl group, preferably an ethyl group, R₃ and R₄ each independently represent a hydrogen atom or a C₁-C₄ alkyl group,

in a solvent such as ethanol, in the presence of sodium acetate, at a temperature of between 50° C. and the reflux temperature of the solvent, for 2 to 20 hours to obtain the compound of formula

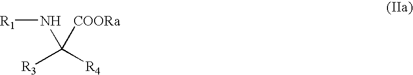

in which R₁, Ra, R₃ and R₄ keep the same meaning as in the starting materials.

The compounds of formula III

R₂—N═C═S (III)

are in general commercial products or can be prepared by following methods which are known to the person skilled in the art, e.g. by reduction of a nitrite compound R₂—NO₂, so as to obtain the primary amine R₂—NH₂, which is then allowed to react for example with thiocarbonyldiimidazole in order to obtain the corresponding isothiocyanate.

It is possible for compounds of formula I in which R₄ represents a hydroxy group to be obtained from compounds of formula (I) in which R₄ is a hydrogen atom, by careful oxidation by means of air oxygen in a solvent such as dimethylsulphoxide (DMSO) for example.

It is possible for compounds of formula I in which one of the R₁ or R₂ groups comprises a primary or secondary amino substituent to be obtained according to a method analogous to methods A and E described above, by using starting materials which bear an amino group protected with an amino-protecting group such as a Boc (t-butyloxycarbonyl) group for example, said protecting group being removed by means known to the person skilled in the art, after obtaining the cyclised compound of central 2-thioxo-4-imidazolidinone structure.

It is possible for compounds of formula I in which X represents an S═O group to be obtained starting from the compounds of formula IIa in which X represents a sulphur atom, by careful oxidation by means for example of a urea/hydrogen peroxide complex, by carrying out the reaction in a solvent such as methanol for example, in the presence of phthalic anhydride, and then a reaction of the ester thus obtained with an isothiocyanate of formula III in accordance with the teaching of method E described above.

Most of the compounds according to the invention have one or more carbon atoms having asymmetry. In the present description, if no indication is specified in the nomenclature, the compound is a racemic compound, i.e. containing R and S isomers in roughly equal amounts. In the case of compounds the asymmetric carbon(s) of which is (are) in a specific configuration, the R or S configuration is indicated corresponding to the position of the substituent introducing the asymmetric centre.

In the examples which follow, the term <<preparation>> designates the examples which describe the synthesis of intermediate compounds, and the term <<Examples>> designates those which describe the synthesis of compounds of formula (I) according to the invention. The aim of these examples is to illustrate the invention: they do not in any way limit the scope of the invention. Melting points are measured on a Koffler block and the spectral values of nuclear magnetic resonance are characterised by the chemical shift calculated with respect to TMS, by the number of protons associated with the signal and by the form of the signal (s for singlet, d for doublet, t for triplet, q for quadruplet, m for multiplet). The working frequency and the solvent used are indicated for each compound.

Preparation I

N-[4-(4-morpholinyl)phenyl]alanine, Ethyl Ester

A solution of 100 g (0.56 M) of 4-(4-morpholinyl)aniline in 3 l of absolute ethanol is prepared, and 69 g (0.84 M) of sodium acetate, then 109 ml (0.84 M) of ethyl 2-bromopropionate, are added. The reaction mixture is then agitated for 16 hours under reflux of the solvent. After cooling, the mixture is filtered and the filtrate is concentrated under reduced pressure. The residue is taken up into 1.5 l of ethyl acetate and the solution obtained is washed with an aqueous solution of sodium chloride. The organic phase is dried over magnesium sulphate, and then concentrated under reduced pressure. The residue is taken up into 0.8 l of isopropyl ether and the solid obtained is isolated by filtration and then dried. 108 g of the product sought after are thus obtained as a fine beige solid (yield=69%).

M PT.=90° C.

Preparation II

N-[4-(4-morpholinyl)phenyl]alanine, Dihydrochloride

A solution of 20 g (71.9 mM) of the ester obtained according to preparation I in 200 ml of tetrahydrofuran is prepared and 84 ml of a normal solution of lithia in water are added. The mixture is agitated for 2 hours at ambient temperature and then the solvent is removed under reduced pressure. The residual aqueous phase is washed 3 times with 100 ml of ethyl ether and then cooled and acidified with 21.6 ml of 10N hydrochloric acid. The mixture is concentrated under reduced pressure until the appearance of crystals. This solid is separated off by filtration and washed on the filter with acetone. After drying, 25.6 g of the product sought after are obtained as a pink solid (the product contains a little lithium chloride).

¹H NMR (DMSO d₆, 300 MHz): 1.38 (d, 3H); 3.48 (m, 4H); 4.05 (m, 4H); 4.07 (q,1H); 6.75 (d,2H); 7.53 (d,2H).

Preparation III

3-[4-[(1,1-dimethylethoxycarbonyl)amino]phenyl]-1-[4-(4-morpholinyl)phenyl]-5-methyl-2-thioxo-4-imidazolidinone

450 mg (1.6 mM) of the ester obtained according to preparation 1 and 410 mg of 4-[(1,1-dimethylethoxycarbonyl)amino]phenyl isothiocyanate are mixed in 10 ml of toluene and 0.4 ml of acetic acid are added. The mixture is agitated at the reflux temperature of the solvent for 5 hours and then cooled to 10-15° C. The white precipitate formed is separated off by filtration, rinsed with 2 ml of cold toluene and then dried under reduced pressure. 720 mg of the product sought after are thus obtained as white crystals (yield=80%).

M PT.=224-226° C.

Preparation IV

3-(trifluoromethoxy)phenyl Isothiocyanate

A solution of 3.46 g (19.5 mM) of 3-(trifluoromethoxy)aniline in 150 ml of dimethylformamide is prepared and is cooled to 0° C. A solution of 3.83 g (21.45 mM) of thiocarbonyldiimidazole dissolved in 60 ml of dimethylformamide is then added dropwise. The reaction mixture is agitated at ordinary temperature for 1 hour 30 minutes, then poured onto 300 ml of water, and extracted with twice 100 ml of ethyl ether. These organic phases are washed with twice 50 ml of water, dried over magnesium sulphate and then concentrated under reduced pressure. This residue is purified by chromatography on silica gel in eluting with the aid of a cyclohexane/ethyl acetate mixture (95/5; v/v). 2.1 g of product sought after are thus obtained as a green-yellow liquid (yield=50%).

¹H NMR (CDCl₃, 300 MHz): 7.38 (t, 1H); 7.15 (m, 3H)

Preparation V

N-[4-(4-morpholinyl)-2-methylphenyl]alanine, Ethyl Ester

In performing analogously to preparation I, starting with 4-(4-morpholinyl)-2-methylaniline, the product sought after is obtained as a yellow powder (yield=78%).

M PT.=70° C.

Preparation VI

N-[3,5-dimethyl-4-(4-morpholinyl)phenyl]alanine, Ethyl Ester

In performing analogously to preparation I, starting with 3,5-dimethyl-4-(4-morpholinyl)aniline, the product sought after is obtained as a beige oil (yield=91%).

¹H NMR (CDCl₃, 300 MHz): 6.25 (s, 2H); 4.20 (m, 3H); 4.07 (m, 1H); 3.75 (t,4H); 3.02 (t,4H); 2.25 (s,6H); 1.49 (d, 3H); 1.28 (t, 3H).

Preparation VII

N-[3,5-dichloro-4-(4-morpholinyl)phenyl]alanine

A mixture of 1.66 g (6.72 mM) of 3,5-dichloro-4-(4-morpholinyl)aniline, 2 g (23.5 mM) of sodium bicarbonate and 1.25 ml (13.44 mM) of 2-bromopropanoic acid is prepared and the reaction mixture is agitated at 100° C. for 4 hours. The mixture is then cooled and then taken up into 60 ml of ethyl acetate and 40 ml of water, and then brought to slightly acidic pH with the aid of an N solution of hydrochloric acid. The separated aqueous phase is extracted with ethyl acetate and the combined organic phases are washed with a solution of sodium chloride and then dried over magnesium sulphate and concentrated under reduced pressure. The crude product thus obtained is used without further purification for the following syntheses.

Preparation VIII

N-[4-(2S,6S-dimethyl-4-morpholinyl)phenyl]alanine, ethyl ester

In performing analogously to preparation I, starting with 4-(2S,6S-dimethyl-4-morpholinyl)aniline, the product sought after is obtained as a yellow oil (yield=87%).

¹H NMR (CDCl₃, 300 MHz): 6.81 (d, 2H); 6.63 (d, 2H); 4.15 (m, 5H); 3.9 (m,1H); 3.08 (2d,2H); 2.75 (2d,2H); 1.48 (d, 3H); 1.32 (d, 6H); 1.30 (t, 3H).

Preparation IX

N-[4-(2R,6S-dimethyl-4-morpholinyl)phenyl]alanine, Ethyl Ester

In performing analogously to preparation I, starting with 4-(2R,6S-dimethyl-4-morpholinyl)aniline, the product sought after is obtained as a pale yellow paste (yield=84%).

¹H NMR (CDCl₃, 300 MHz): 6.82 (d, 2H); 6.59 (d, 2H); 4.17 (q, 2H); 4.07 (m, 1H); 3.85 (m, 3H); 3.25 (d, 2H); 2.33 (t, 2H); 1.45 (d, 3H); 1.24 (t, 3H); 1.23 (d, 6H).

Preparation X

2-methyl-N-[4-(4-morpholinyl)phenyl]alanine, Ethyl Ester

In performing analogously to preparation I, starting with ethyl 2-bromo-2-methylpropanoate, the product sought after is obtained as beige crystals (yield=70%).

M PT.=78° C.

Preparation XI

1-(4-nitrophenyl)-4-piperidinemethanol

A solution of 1.4 g (10 mM) of 4-fluoro-1-nitrobenzene in 20 ml of dimethylsulphoxide is prepared and 2.5 g (22 mM) of 4-piperidinemethanol are added. The reaction mixture is kept under agitation for 1 hour at 80° C. and then cooled and poured onto 200 ml of water. The yellow precipitate formed is separated off by filtration, washed with water and dried. 2.3 g of the product sought after are thus obtained as a white powder (yield=99%).

M PT.=161° C.

Preparation XII

1-(4-aminophenyl)-4-piperidinemethanol

A solution of 2.3 g of the compound obtained according to preparation XI in 150 ml of methanol is prepared and 200 mg de 10% palladium on carbon are added. The mixture is agitated under a hydrogen atmosphere for 1 hour 30 minutes, under atmospheric pressure and at ambient temperature. The catalyst is then separated off by filtration and the filtrate is concentrated under reduced pressure. 2 g of the product sought after are thus obtained as a beige powder (yield=99%).

M PT.=105° C.

Preparation XIII

N-[4-[4-(hydroxymethyl)-1-piperidinyl]phenyl]alanine, Dihydrochloride

A solution of 1.95 g of the compound obtained according to preparation XII and 2 ml of 2-bromopropanoic acid is prepared and 2.78 g (33.2 mM) of sodium bicarbonate are added. The reaction mixture is kept under agitation for 30 minutes at 100° C., and then cooled and dissolved in 100 ml of water. The solution is acidified to pH 1 with the aid of hydrochloric acid and this aqueous phase is washed with 50 ml of dichloromethane and then concentrated under reduced pressure. 3.9 g of the non-purified acid sought after are thus obtained, as beige crystals which are used directly in the next step without other purification.

Preparation XIV

N-[4-(4-thiomorpholinyl)phenyl]alanine, Ethyl Ester

In performing analogously to preparation I, starting with 4-(4-thiomorpholinyl)aniline, the product sought after is obtained as a white powder (yield=48%).

M PT.=240° C.

Preparation XV

N-[4-(4-thiomorpholinyl)phenyl]alanine, Ethyl Ester, S-Oxide

A solution of 0.13 g (1.36 mM) of the urea/hydrogen peroxide addition compound in 4 ml of methanol is prepared and 0.05 g (0.34 mM) of phthalic anhydride, and then 0.2 g (0.68 mM) of the ester obtained according to preparation XIV, are added. The reaction mixture is kept under agitation for 1 hour 30 minutes at ambient temperature, and then poured onto 50 ml of water. The mixture is extracted with twice 50 ml of ethyl acetate and the combined organic phases are then washed with water and then dried over magnesium sulphate and concentrated under reduced pressure. The residue is purified by chromatography on silica gel in eluting with the aid of a dichloromethane/methanol mixture (99/1; v/v). 80 mg of the product sought after are thus obtained (yield=38%).

Preparation XVI

N-[4-(4-morpholinyl)phenyl]glycine, Dihydrochloride

10 g (57 mM) of 4-(4-morpholinyl)aniline and 16.5 g of sodium bicarbonate are mixed well. 9.4 g (67 mM) of bromoacetic acid are added. The mixture is agitated at 120° C. for 6 minutes and then cooled and poured onto 100 ml of water. The aqueous phase obtained is washed with 50 ml of dichloromethane and then slowly acidified to pH 1 with hydrochloric acid. The aqueous phase is concentrated under reduced pressure and the solid residue is triturated with 100 ml of a dichloromethane/methanol mixture (80/20; v/v). The mixture is filtered and the filtrate concentrated under reduced pressure enables 16 g of brown crystals to be obtained which are used without further purification for the next step.

Preparations XVII to LXXX relating to novel intermediates which are useful for the synthesis of compounds of formula (I), and which are in general obtained according to methods analogous to those of the preceding preparations or according to methods described further on (such as method P), are grouped in Table II, situated further on.

### EXAMPLE 1

3-(4-methoxyphenyl)-5-methyl-1-[4-(4-morpholinyl)phenyl]-2-thioxo-4-imidazolidinone

A solution of 45 g (0.16 M) of the compound obtained according to preparation I in 400 ml of toluene is prepared and 36.3 g (0.22 M) of 4-(isothiocyanato)anisole, and then 20 ml of acetic acid, are added. The reaction mixture is then kept under reflux for 16 hours. The reaction medium is concentrated under reduced pressure and the residue is purified by chromatography on silica gel in eluting with the aid of a toluene/ethyl acetate mixture (80/20; v/v). 53 g of the product sought after are thus obtained as a pale yellow solid (yield=82.5%).

M PT.=181° C.

### EXAMPLE 2

5-methyl-1-[4-(4-morpholinyl)phenyl]-3-phenyl-2-thioxo-4-imidazolidinone

In performing analogously to Example 1, starting with phenyl isothiocyanate, the product sought after is obtained as a pale yellow powder (yield=77%).

M PT.=214° C.

### EXAMPLE 3

5-methyl-1-[4-(4-morpholinyl)phenyl]-3-phenyl-2-thioxo-4-imidazolidinone, Hydrochloride

1 g (2.72 mM) of the compound obtained according to Example 2 is dissolved in 5 ml of dichloromethane. The solution is cooled to 0° C. and then 1.3 ml of a saturated ethylic solution of hydrogen chloride are added. The white precipitate is separated off by filtration, washed with a little ethyl ether and dried under reduced pressure. 1.1 g of the product sought after are thus obtained as a white powder (yield=99%).

M PT.=212° C.

### EXAMPLE 4

3-(4-hydroxyphenyl)-5-methyl-1-[4-(4-morpholinyl)phenyl]-2-thioxo-4-imidazolidinone

In performing analogously to Example 1, starting with 4-(isothiocyanato)-phenol, the product sought after is obtained as a white powder (yield=52%).

M PT.=220° C.

### EXAMPLE 5

5-methyl-3-(3-methoxyphenyl)-1-[4-(4-morpholinyl)phenyl]-2-thioxo-4-imidazolidinone

In performing analogously to Example 1, starting with 3-methoxyphenyl isothiocyanate, the product sought after is obtained as beige crystals (yield=58%).

M PT.=175° C.

### EXAMPLE 6

3-(4-ethoxyphenyl)-5-methyl-1-[4-(4-morpholinyl)phenyl]-2-thioxo-4-imidazolidinone

In performing analogously to Example 1, starting with 4-ethoxyphenyl isothiocyanate, the product sought after is obtained as white crystals with a yield of 48%.

M PT.=180-182° C.

### EXAMPLE 7

3-(4-chlorophenyl)-5-methyl-1-[4-(4-morpholinyl)phenyl]-2-thioxo-4-imidazolidinone

0.6 g (2 mM) of the acid obtained according to preparation II are dissolved in 5 ml of dichloromethane and 0.1 g of triethylamine and 0.68 g (4 mM) of 4-chlorophenyl isothiocyanate are added. The reaction mixture is kept under agitation for 20 hours at ambient temperature, and then concentrated under reduced pressure. The residue is purified by chromatography on silica gel in eluting with the aid of a dichloromethane/ethyl acetate mixture (96/4; v/v). 0.37 g of the product sought after are thus obtained as a white powder (yield=46%).

M PT.=212° C.

### EXAMPLE 8

3-(3-chlorophenyl)-5-methyl-1-[4-(4-morpholinyl)phenyl]-2-thioxo-4-imidazolidinone

In performing analogously to Example 1, starting with 3-chlorophenyl isothiocyanate, the product sought after is obtained as beige crystals (yield=54%).

M PT.=137-138° C.

### EXAMPLE 9

3-(2-chlorophenyl)-5-methyl-1-[4-(4-morpholinyl)phenyl]-2-thioxo-4-imidazolidinone

In performing analogously to Example 7, starting with 2-chlorophenyl isothiocyanate, the product sought after is obtained as yellow crystals (yield=35%).

M PT.=116° C.

### EXAMPLE 10

3-(4-fluorophenyl)-5-methyl-1-[4-(4-morpholinyl)phenyl]-2-thioxo-4-imidazolidinone

In performing analogously to Example 1, starting with 4-fluorophenyl isothiocyanate, the product sought after is obtained as white crystals (yield=52%).

M PT.=188-190° C.

### EXAMPLE 11

3-(3-fluorophenyl)-5-methyl-1-[4-(4-morpholinyl)phenyl]-2-thioxo-4-imidazolidinone

In performing analogously to Example 1, starting with 3-fluorophenyl isothiocyanate, the product sought after is obtained as cream-coloured crystals (yield=74%).

M PT.=196-198 C

### EXAMPLE 12

3-(2-fluorophenyl)-5-methyl-1-[4-(4-morpholinyl)phenyl]-2-thioxo-4-imidazolidinone

In performing analogously to Example 1, starting with 2-fluorophenyl isothiocyanate, the product sought after is obtained as yellow crystals (yield=58%).

M PT.=186-188° C.

### EXAMPLE 13

5-methyl-3-(3-methylphenyl)-1-[4-(4-morpholinyl)phenyl]-2-thioxo-4-imidazolidinone

In performing analogously to Example 1, starting with 3-methylphenyl isothiocyanate, the product sought after is obtained as beige crystals (yield=46%).

M PT.=160-162° C.

### EXAMPLE 14

5-methyl-3-(2-methylphenyl)-1-[4-(4-morpholinyl)phenyl]-2-thioxo-4-imidazolidinone

In performing analogously to Example 1, starting with 2-methylphenyl isothiocyanate, the product sought after is obtained as white crystals (yield=9%).

M PT.=143-145° C.

### EXAMPLE 15

5-methyl-1-[4-(4-morpholinyl)phenyl]-3-(4-nitrophenyl)-2-thioxo-4-imidazolidinone

In performing analogously to Example 1, starting with 4-nitrophenyl isothiocyanate, the product sought after is obtained as yellow crystals (yield=88%).

M PT.=208-210° C.

### EXAMPLE 16

3-(4-aminophenyl)-5-methyl-1-[4-(4-morpholinyl)phenyl]-2-thioxo-4-imidazolidinone

500 mg of the compound obtained according to preparation III are dissolved in 90 ml of dichloromethane, 10 ml of trifluoroacetic acid are added and then this mixture is agitated for one hour at 20° C. The reaction mixture is then concentrated under reduced pressure and the residue is taken up into suspension in 100 ml of a saturated solution of sodium bicarbonate. This suspension is extracted with dichloromethane and the organic phase obtained is concentrated under reduced pressure. The residue is purified by chromatography on silica gel in eluting with the aid of a dichloromethane/methanol mixture (96/4; v/v). 400 mg of the product sought after are thus obtained as white crystals (yield=95%).

M PT.=269-270° C.

### EXAMPLE 17

5-methyl-3-[4-(methylthio)phenyl]-1-[4-(4-morpholinyl)phenyl]-2-thioxo-4-imidazolidinone

In performing analogously to Example 1, starting with 4-(methylthio)phenyl isothiocyanate, the product sought after is obtained as cream-coloured crystals (yield=77%).

M PT.=168-170° C.

### EXAMPLE 18

5-methyl-3-[4-(1-methylethoxy)phenyl]-1-[4-(4-morpholinyl)phenyl]-2-thioxo-4-imidazolidinone

In performing analogously to Example 1, starting with 4-(1-methylethoxy)phenyl isothiocyanate, the product sought after is obtained as a cream-coloured powder (yield=60%).

M PT.=120-122 C

### EXAMPLE 19

5-methyl-1-[4-(4-morpholinyl)phenyl]-2-thioxo-3-[3-(trifluoromethoxy)-phenyl]-4-imidazolidinone

In performing analogously to Example 1, starting with 3-(trifluoromethoxy)phenyl isothiocyanate, the product sought after is obtained as a brown powder (yield=56%).

M PT.=84-88° C.

### EXAMPLE 20

5-methyl-1-[4-(4-morpholinyl)phenyl]-2-thioxo-3-[3-(trifluoromethyl)-phenyl]-4-imidazolidinone

In performing analogously to Example 1, starting with 3-(trifluoromethyl)phenyl isothiocyanate, the product sought after is obtained as cream-coloured crystals (yield=70%).

M PT.=163-165° C.

### EXAMPLE 21

3-(3,4-dimethoxyphenyl)-5-methyl-1-[4-(4-morpholinyl)phenyl]-2-thioxo-4-imidazolidinone

In performing analogously to Example 1, starting with 3,4-(dimethoxy)phenyl isothiocyanate, the product sought after is obtained as a pale yellow fluffy solid (yield=35%).

M PT.=214-216° C.

### EXAMPLE 22

3-(2,4-dimethoxyphenyl)-5-methyl-1-[4-(4-morpholinyl)phenyl]-2-thioxo-4-imidazolidinone

In performing analogously to Example 1, starting with 2,4-(dimethoxy)phenyl isothiocyanate, the product sought after is obtained as orange crystals (yield=31%).

M PT.=110° C.

### EXAMPLE 23

5-methyl-3-(3,4-methylenedioxyphenyl)-1 [4-(morpholinyl)phenyl]-2-thioxo-4-imidazolidinone

In performing analogously to Example 1, starting with 3,4-(methylenedioxy)phenyl isothiocyanate, the product sought after is obtained as a yellow fluffy solid (yield=55%).

M PT.=223-225° C.

### EXAMPLE 24

3-(4-methoxy-2-nitrophenyl)-5-methyl-1-[4-(4-morpholinyl)phenyl]-2-thioxo-4-imidazolidinone

In performing analogously to Example 1, starting with 4-methoxy-2-nitrophenyl isothiocyanate, the product sought after is obtained as beige crystals (yield=56%).

M PT.=178-180° C.

### EXAMPLE 25

3-(4-methoxy-2-methylphenyl)-5-methyl-1-[4-(4-morpholinyl)phenyl]-2-thioxo-4-imidazolidinone

In performing analogously to Example 7, starting with 4-methoxy-2-methylphenyl isothiocyanate, the product sought after is obtained as cream-coloured crystals (yield=12%).

M PT.=144-146° C.

### EXAMPLE 26

3-(3,4-difluorophenyl)-5-methyl-1-[4-(4-morpholinyl)phenyl]-2-thioxo-4-imidazolidinone

In performing analogously to Example 1, starting with 3,4-difluorophenyl isothiocyanate, the product sought after is obtained as a white powder (yield=62%).

M PT.=164-165° C.

### EXAMPLE 27

5-methyl-1-[4-(4-morpholinyl)phenyl]-3-(3-pyridinyl)-2-thioxo-4-imidazolidinone

In performing analogously to Example 1, starting with 3-pyridinyl isothiocyanate, the product sought after is obtained as cream-coloured crystals (yield=15%).

M PT.=152-154° C.

### EXAMPLE 28

5-methyl-1-[4-(4-morpholinyl)phenyl]-3-(2-thienyl)-2-thioxo-4-imidazolidinone

In performing analogously to Example 1, starting with 2-thienyl isothiocyanate, the product sought after is obtained as a beige powder (yield=35%).

M PT.=184-185° C.

### EXAMPLE 29

3-ethyl-5-methyl-1-[4-(4-morpholinyl)phenyl]-2-thioxo-4-imidazolidinone

In performing analogously to Example 1, starting with ethyl isothiocyanate, the product sought after is obtained as a yellow powder (yield=61%).

M PT.=126° C.

### EXAMPLE 30

5-methyl-1-[4-(4-morpholinyl)phenyl]-3-(2-propenyl)-2-thioxo-4-imidazolidinone

In performing analogously to Example 1, starting with 2-propenyl isothiocyanate, the product sought after is obtained as an off-white powder (yield=54%).

M PT.=106° C.

### EXAMPLE 31

3-(cyclopentyl)-5-methyl-1-[4-(4-morpholinyl)phenyl]-2-thioxo-4-imidazolidinone

In performing analogously to Example 1, starting with cyclopentyl isothiocyanate, the product sought after is obtained as a white solid (yield=41%).

M PT.=148-149° C.

### EXAMPLE 32

5-methyl-1-[4-(4-morpholinyl)-2-methylphenyl]-3-phenyl-2-thioxo-4-imidazolidinone

In performing analogously to Example 2, starting with the ester obtained according to preparation V, the product sought after is obtained as a beige powder (yield=36%).

M PT.=180° C.

### EXAMPLE 33

1-[3,5-dimethyl-4-(4-morpholinyl)phenyl]-3-(4-methoxyphenyl)-5-methyl-2-thioxo-4-imidazolidinone

In performing analogously to Example 1, starting with the ester obtained according to preparation VI, the product sought after is obtained as an off-white powder (yield=48%).

M PT.=240 C

### EXAMPLE 34

1-[3,5-dichloro-4-(4-morpholinyl)phenyl]-5-methyl-3-phenyl-2-thioxo-4-imidazolidinone

In performing analogously to Example 7, starting with the acid obtained according to preparation VII, the product sought after is obtained as a white powder (yield=16%).

M PT.=255° C.

### EXAMPLE 35

1-[4-(2S,6S-dimethyl-4-morpholinyl)phenyl]-5-methyl-3-phenyl-2-thioxo-4-imidazolidinone

In performing analogously to Example 2, starting with the ester obtained according to preparation VIII, the product sought after is obtained as a white powder (yield=80%).

M PT.=184° C.

### EXAMPLE 36

1-[4-(2R,6S-dimethyl-4-morpholinyl)phenyl]-5-methyl-3-phenyl-2-thioxo-4-imidazolidinone

In performing analogously to Example 2, starting with the ester obtained according to preparation IX, the product sought after is obtained as a white powder (yield=85%).

M PT.=200° C.

### EXAMPLE 37

1-[4-(2R,6S-dimethyl-4-morpholinyl)phenyl]-3-(4-methoxyphenyl)-5-methyl-2-thioxo-4-imidazolidinone

In performing analogously to Example 1, starting with the ester obtained according to preparation IX, the product sought after is obtained as a pale yellow powder (yield=63%).

M PT.=210° C.

### EXAMPLE 38

1-[4-(2R,6S-dimethyl-4-morpholinyl)phenyl]-3-(3-fluorophenyl)-5-methyl-2-thioxo-4-imidazolidinone

In performing analogously to Example 37, starting with 3-fluorophenyl isothiocyanate, the product sought after is obtained as a white powder (yield=96%).

M PT.=217° C.

### EXAMPLE 39

5,5-dimethyl-1-[4-(4-morpholinyl)phenyl]-3-phenyl-2-thioxo-4-imidazolidinone

In performing analogously to Example 2, starting with the ester obtained according to preparation X, the product sought after is obtained as a beige powder (yield=23%).

M PT.=206° C.

### EXAMPLE 40

5,5-dimethyl-3-(4-methoxyphenyl)-1-[4-(4-morpholinyl)phenyl]-2-thioxo-4-imidazolidinone

In performing analogously to Example 1, starting with the ester obtained according to preparation X, the product sought after is obtained as a white powder (yield=30%).

M PT.=225-230° C.

### EXAMPLE 41

5,5-dimethyl-3-(3-fluorophenyl)-1-[4-(4-morpholinyl)phenyl]-2-thioxo-4-imidazolidinone

In performing analogously to Example 11, starting with the ester obtained according to preparation X, the product sought after is obtained as a beige powder (yield=60%).

M PT.=219° C.

### EXAMPLE 42

3-(3-chlorophenyl)-5,5-dimethyl-1-[4-(4-morpholinyl)phenyl]-2-thioxo-4-imidazolidinone

In performing analogously to Example 8, starting with the ester obtained according to preparation X, the product sought after is obtained as white crystals (yield=32%).

M PT.=220° C.

### EXAMPLE 43

5,5-dimethyl-3-(3,4-methylenedioxyphenyl)-1-[4-(4-morpholinyl)phenyl]-2-thioxo-4-imidazolidinone

In performing analogously to Example 23, starting with the ester obtained according to preparation X, the product sought after is obtained as white crystals (yield=24%).

M PT.=202° C.

### EXAMPLE 44

1-[4-[4-(hydroxymethyl)-1-piperidinyl]phenyl}-3-(4-methoxyphenyl)-5-methyl-2-thioxo-4-imidazolidinone

A solution of 1 g (3.6 mM) of the amino acid obtained according to preparation XIII in 20 ml of acetonitrile is prepared and 0.75 ml (5.4 mM) of 4-methoxyphenyl isothiocyanate are added, and then 2 ml (14.4 mM) of triethylamine. The reaction mixture is kept under agitation for 2 hours at ambient temperature and then concentrated under reduced pressure. The residue is taken up into 50 ml of water and 100 ml of dichloromethane. The separated organic phase is dried over magnesium sulphate and then concentrated under reduced pressure. The residue is purified by chromatography on silica gel in eluting with the aid of a dichloromethane/methanol mixture (95/5; v/v). 370 mg of the product sought after are thus obtained as a white powder (yield=25%)

M PT.=88-90° C.

### EXAMPLE 45

5-hydroxy-5-methyl-1-[4-(4-morpholinyl)phenyl]-3-phenyl-2-thioxo-4-imidazolidinone

A solution of 1.7 g (4.3 mM) of the compound obtained according to Example 2 in 85 ml of dimethylsulphoxide is prepared and 8.5 ml of water are added. The reaction mixture is kept for 22 hours at 100° C., with introduction of compressed air. The solution is then cooled, poured onto 850 ml of water and the mixture obtained is extracted several times with ethyl acetate. The combined organic phases are washed with a solution of sodium chloride and then dried over magnesium sulphate and concentrated under reduced pressure. The residue is purified by chromatography on silica gel in eluting with the aid of a dichloromethane/ethyl ether mixture (90/10; v/v). The crystals obtained are washed with cyclohexane and then dried. 0.54 g of the product sought after are thus obtained as cream crystals (yield=54%).

M PT.=242-244° C.

### EXAMPLE 46

5-methyl-3-phenyl-1-[4-(4-thiomorpholinyl)phenyl]-2-thioxo-4-imidazolidinone, S-oxide

In performing analogously to Example 2, starting with the compound obtained according to preparation XV, the product sought after is obtained as white crystals (yield=55%).

M PT.=230° C.

### EXAMPLE 47

3-(3,4-dimethoxyphenyl)-5,5-dimethyl-1-[4-(4-morpholinyl)phenyl]-2-thioxo-4-imidazolidinone

In performing analogously to Example 39, starting with 3,4-dimethoxyphenyl isothiocyanate, the product sought after is obtained as white crystals (yield=7%).

M PT.=180° C.

### EXAMPLE 48

5-hydroxy-3-(4-methoxy-2-methylphenyl)-5-methyl-1-[4-(4-morpholinyl)-phenyl]-2-thioxo-4-imidazolidinone

1 g (2.67 mM) of the amino acid obtained according to preparation II are mixed with 0.83 ml (5.34 mM) of 4-methoxy-2-methylphenyl isothiocyanate and 1.1 ml of triethylamine in 30 ml of dichloromethane and 30 ml of methanol are added. The reaction mixture is agitated for 24 hours at ambient temperature and then concentrated under reduced pressure. The residue is purified by chromatography on silica gel in eluting with the, aid off a dichloromethane/ethyl ether mixture (80/20; v/v). 0.23 g of the product sought are thus obtained after as a white powder (yield=21%).

M PT.=205° C.

### EXAMPLE 49

1-[4-(4-morpholinyl)phenyl]-3-phenyl-2-thioxo-4-imidazolidinone

8 g of the acid obtained according to preparation XVI, 8 ml (68 mM) of phenyl isothiocyanate and 19 ml of triethylamine are mixed in 100 ml of acetonitrile and the mixture is agitated for 16 hours at ambient temperature. The reaction medium is then concentrated under reduced pressure and the residue is purified by chromatography on silica gel in eluting with a toluene/ethyl acetate mixture (60/40; v/v). 250 mg of the product sought after are thus obtained as beige crystals (yield=2%).

M PT.=250° C.

### EXAMPLE 50

3-[4-(4-morpholinyl)phenyl]-5-methyl-1-phenyl-2-thioxo-4-imidazolidinone

In performing analogously to Example 1, starting with the ethyl ester of N-phenylalanine and 4-(4-morpholinyl)phenyl isothiocyanate, the product sought after is obtained as a white powder (yield=64%).

M PT.=201 C

The chemical structures of the compounds according to the invention described above are summarised in Table I.

The other novel compounds, intermediates or compounds according to the invention, which are obtained according to methods analogous to those described above are grouped in the following Tables in which the chemical structure, certain physical characteristics, the yield of the reaction (noted as <<yld>>) and the preparation method, can be found. The melting point (M PT) is expressed in ° C.

Table III groups other examples of compounds according to the invention, which are in general obtained according to methods analogous to those described above.

In the case of salified compounds, HCl signifies hydrochloride, HBr signifies hydrobromide, Sulph signifies sulphate, Ms signifies methanesulphonate, Tfa signifies trifluoroacetate.

The compounds appearing in these Tables are obtained by means of methods analogous to those of the Preparations or Examples described above (method A is analogous to Example 7, method E is analogous to Example 1) or according to the methods described below (method M with microwaves, method F by fusion without solvent, method S with in situ generation of the isothiocyanate and method P of preparation of an amino ester).

Methods of obtaining intermediates or compounds of formula I:

Method M: (General Method)

1 mmole of ester of formula (IIa) and 1.2 mmole of isothiocyanate R₂—NCS (III) are placed in a PTFE reactor, and 2 drops of acetic acid are added. The reactor is then placed in a domestic microwave oven and is irradiated for 2 to 10 minutes (e.g. 2 minutes when R₃=CH₃ and R₄═H, and 10 minutes when R₃=R₄=CH₃), under a power of 700 to 900 W. After irradiation, the reactor is cooled and the reaction mixture is taken up with about 20 ml of ethyl ether. If the product sought after crystallises, the mixture is filtered and the compound sought after is isolated. If the product sought after does not crystallise, or is obtained impure, a purification by chromatography on silica gel is effected so as to obtain the pure product. The yields are indicated in the recapitulative Table of the compounds according to the invention.

Method F (Example 62):

The compound obtained according to preparation XXII (0.5 g; 1.71 mM) is mixed well with 0.35 g (2.05 mM) of 2,5-difluorophenyl isothiocyanate. After adding 5 drops of acetic acid, the reaction mixture is brought to a temperature of 120° C. (oil bath) for 1 hour 30 minutes. The product of the reaction is purified directly by chromatography on silica gel in eluting With the aid of a dichloromethane/ethyl acetate mixture (97/3; v/v). After crystallisation in isopropyl ether, the product sought after is obtained as a white solid (yield: 80%).

M PT.=148° C.

Method P (Preparation LXIII):

A solution of 0.3 g (1.27 mM) of 2,6-dimethyl-4-(4-morpholinyl)nitrobenzene in 15 ml of ethanol is prepared in a Parr flask. 0.217 g (1.27 mM) of sodium sulphate, 0.56 ml (1.27 mM) of ethyl pyruvate are added successively and under a nitrogen atmosphere. 30 mg of 10% palladium on carbon are finally added. The mixture obtained is hydrogenated under agitation and under a pressure of 3,400 hPa at ambient temperature for 5 hours. The reaction mixture is then filtered and the filtrate is concentrated under reduced pressure. The residue from evaporation is purified by chromatography on silica gel in eluting with the aid of a hexane/ethyl acetate mixture (80/20; v/v). The product sought after is obtained as a yellow oil (yield: 57%).

Method S (Example 303):

A solution of 1 g (5.6 mM) of thiocarbonyldiimidazole in 20 ml of dichloromethane is prepared and a solution of 1 g (5.6 mM) of 4-(4-morpholinyl)aniline in 10 ml of dichloromethane is added dropwise. The reaction mixture is then agitated for 1 hour at ambient temperature. 1.09 g (5.6 mM) of N-(4-methoxyphenyl)alanine in 10 ml of dichloromethane, then 0.78 ml (5.6 mM) of triethylamine, are added. The reaction mixture is agitated for 4 hours and then concentrated under reduced pressure. The residue from evaporation is purified by chromatography on silica gel in eluting with the aid of a dichloromethane/ethyl acetate mixture (90/10; v/v). The product sought after is obtained as white crystals (yield: 54%).

M PT.=202° C.

TABLE I

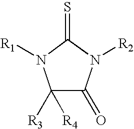

| EX | R₁ | R₂ | R₃ | R₄ |
|---|---|---|---|---|
| 1 | 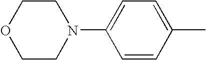 | 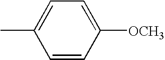 | CH₃ | H |
| 2 | 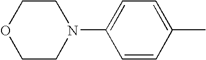 | 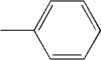 | CH₃ | H |
| 3* | 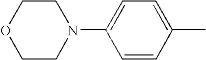 | 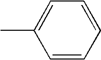 | CH₃ | H |
| 4 | 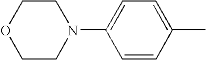 | 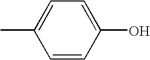 | CH₃ | H |
| 5 | 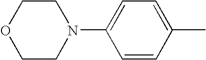 | 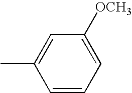 | CH₃ | H |
| 6 | 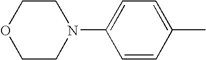 | 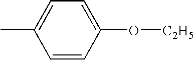 | CH₃ | H |
| 7 | 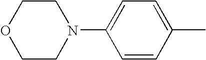 | 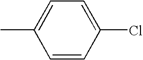 | CH₃ | H |
| 8 | 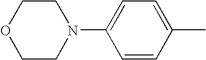 | 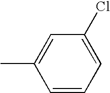 | CH₃ | H |
| 9 | 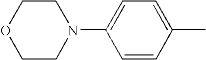 | 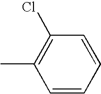 | CH₃ | H |
| 10 | 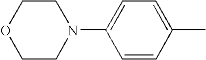 | 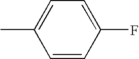 | CH₃ | H |
| 11 | 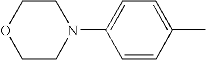 | 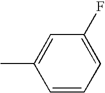 | CH₃ | H |
| 12 | 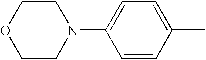 | 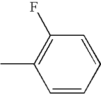 | CH₃ | H |
| 13 | 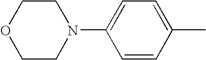 | 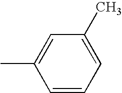 | CH₃ | H |
| 14 | 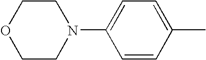 | 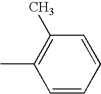 | CH₃ | H |
| 15 | 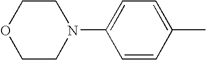 | 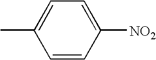 | CH₃ | H |
| 16 | 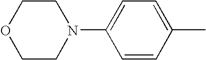 | 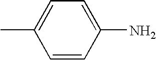 | CH₃ | H |
| 17 | 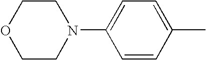 | 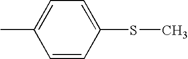 | CH₃ | H |
| 18 | 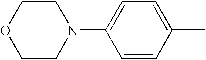 | 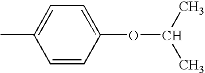 | CH₃ | H |
| 19 | 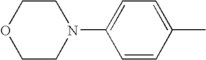 | 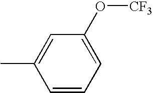 | CH₃ | H |
| 20 | 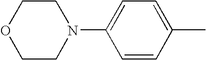 | 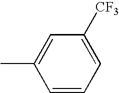 | CH₃ | H |
| 21 | 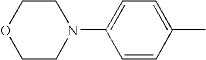 | 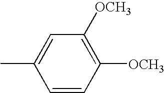 | CH₃ | H |
| 22 | 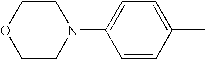 | 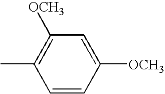 | CH₃ | H |
| 23 | 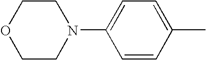 | 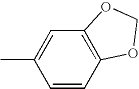 | CH₃ | H |
| 24 | 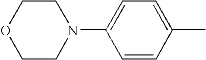 | 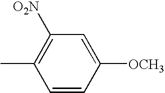 | CH₃ | H |
| 25 | 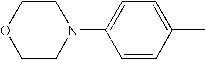 | 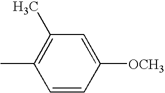 | CH₃ | H |
| 26 | 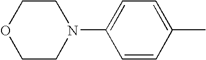 | 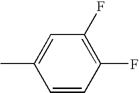 | CH₃ | H |
| 27 | 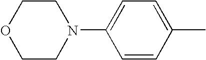 | 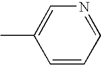 | CH₃ | H |
| 28 | 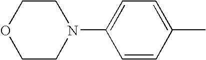 | 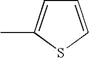 | CH₃ | H |
| 29 | 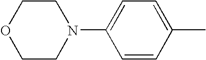 | —C₂H₅ | CH₃ | H |
| 30 | 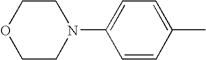 | —CH₂—CH═CH₂ | CH₃ | H |
| 31 | 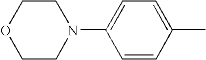 | 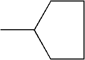 | CH₃ | H |
| 32 | 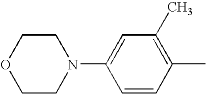 | 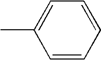 | CH₃ | H |
| 33 | 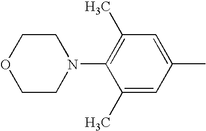 | 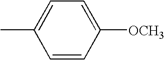 | CH₃ | H |
| 34 | 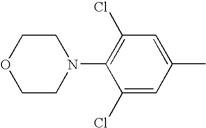 | 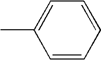 | CH₃ | H |
| 35 | 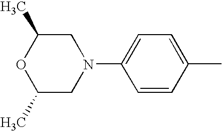 | 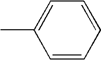 | CH₃ | H |
| 36 | 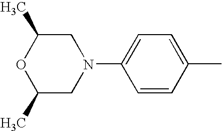 | 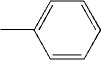 | CH₃ | H |
| 37 | 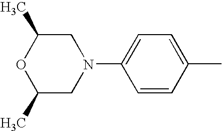 | 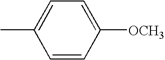 | CH₃ | H |
| 38 | 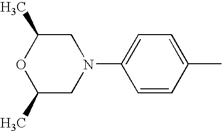 | 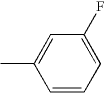 | CH₃ | H |
| 39 | 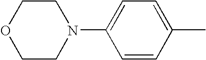 | 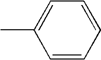 | CH₃ | CH₃ |
| 40 | 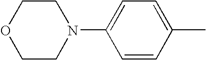 | 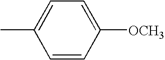 | CH₃ | CH₃ |
| 41 | 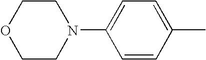 | 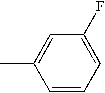 | CH₃ | CH₃ |
| 42 | 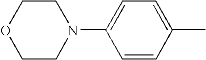 | 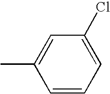 | CH₃ | CH₃ |
| 43 | 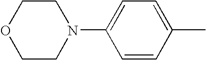 | 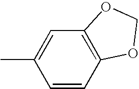 | CH₃ | CH₃ |
| 44 | 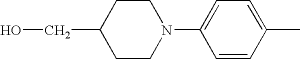 | 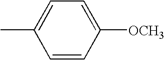 | CH₃ | H |
| 45 | 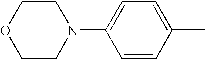 | 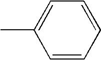 | CH₃ | OH |
| 46 | 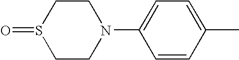 | 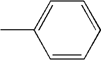 | CH₃ | H |
| 47 | 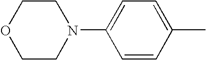 | 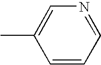 | CH₃ | CH₃ |
| 48 | 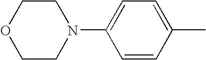 | 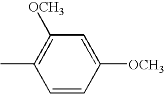 | CH₃ | OH |
| 49 | 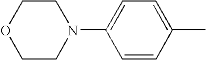 | 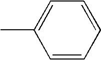 | H | H |
| 50 | 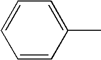 | 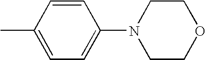 | CH₃ | H |

*: hydrochloride of Example 2

TABLE II

| Preparation | | M PT | | Yld | Method |
|---|---|---|---|---|---|
| No. | Structure | ° C. | Appearance | % | (*) |
| XVII | 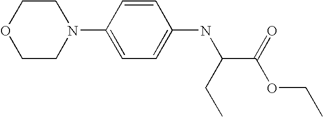 | 107 | Solid yellow | 66 | I |
| XVIII | 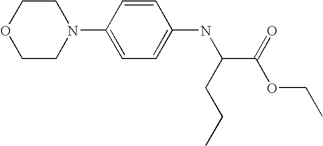 | 98 | Beige powder | 67 | I |
| XIX | 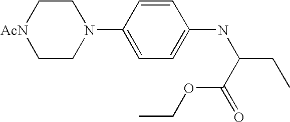 | 123 | Beige solid | 53 | I |
| XX | 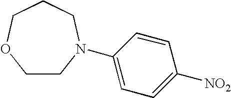 | 98 | Yellow powder | 89 | XI |
| XXI | 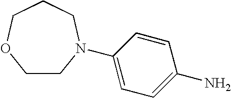 | NMR | Violet oil | 97 | XII |
| XXII | 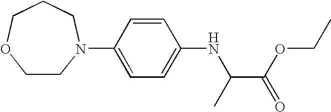 | NMR | light brown oil | 64 | I |
| XXIII | 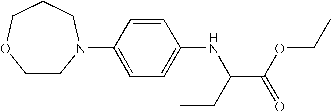 | NMR | Yellow oil | 88 | I |
| XXIV | 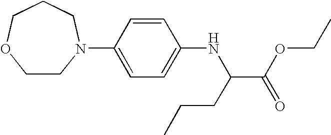 | NMR | Yellow oil | 91 | I |
| XXV | 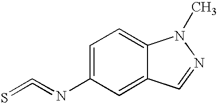 | 81 | Brown powder | 100 | IV |
| XXVI | 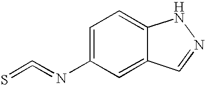 | >260 | Yellow powder | 30 | IV |
| XXVII | 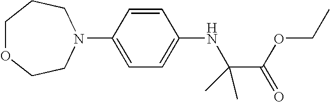 | NMR | Yellow oil | 79 | I |
| XXVIII | 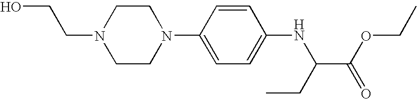 | NMR | Brown oil | 59 | I |
| XXIX | 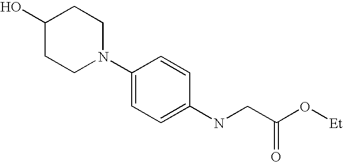 | | Brown oil | 52 | I |
| XXX | 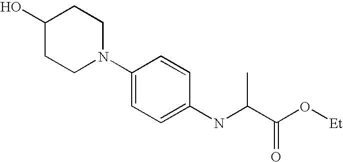 | 60-70 | gum | 84 | I |
| XXXI | 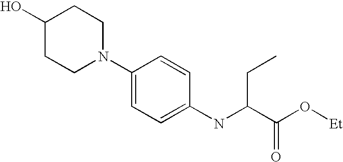 | NMR | Black oil | 7 | I |
| XXXII | 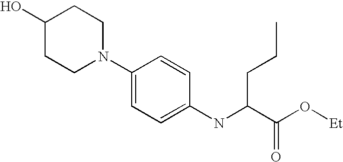 | NMR | Black oil | 91 | I |
| XXXV | 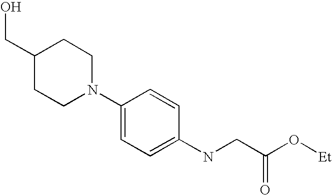 | 61 | White crystals | 62 | I |
| XXXVI | 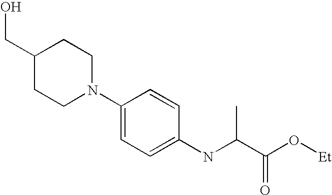 | 92-94 | White crystals | 62 | I |
| XXXVII | 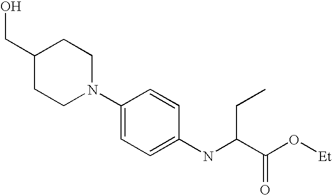 | 90-92 | White crystals | 57 | I |
| XXXVIII | 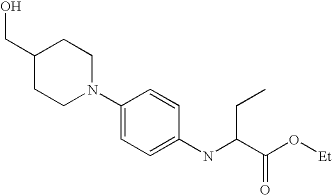 | 58-60 | Beige crystals | 46 | I |
| XXXIX | 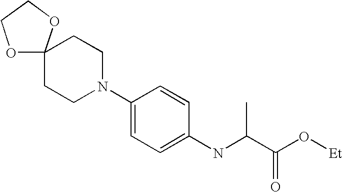 | 81 | light brown solid | 76 | I |
| XL | 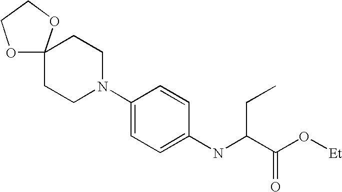 | 60 | Yellow solid | 72 | I |
| XLI | 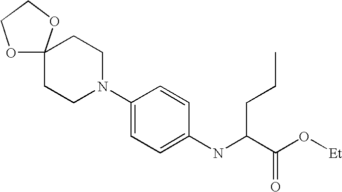 | NMR | Yellow oil | 63 | I |
| XLIII | 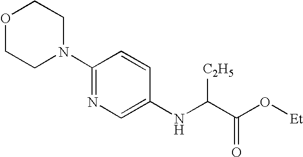 | 67 | Violet solid | 92 | I |
| XLIV | 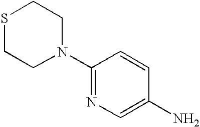 | NMR | Violet oil | 90 | XII |
| XLV | 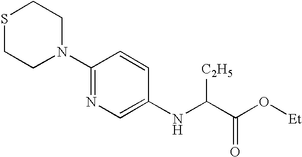 | NMR | Violet oil | 72 | I |
| XLVI | 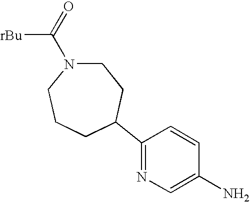 | NMR | Violet foam | 100 | XII |
| XLVII | 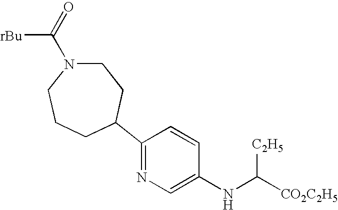 | NMR | Violet oil | 92 | I |
| XLVIII | 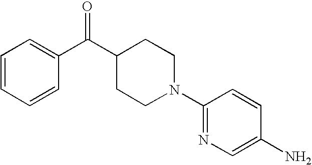 | 146 | Purplis pink powder | 60 | XII |
| IL | 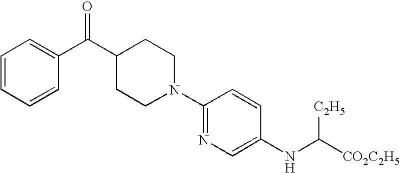 | NMR | Violet oil | 72 | I |
| L | 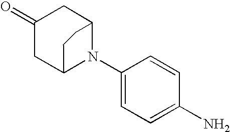 | 159 | Brown solid | 65 | XII |
| LI | 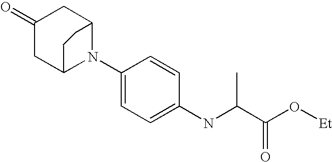 | 93 | Beige solid | 49 | I |
| LII | 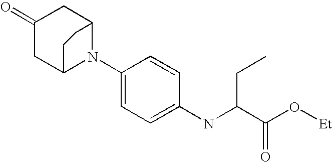 | NMR | Sticky solid | 42 | I |
| LIII | 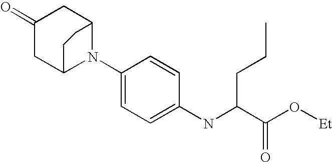 | NMR | Sticky solid | 42 | I |
| LIV | 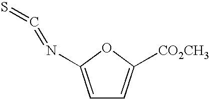 | NMR | Brown oil | 8 | IV |
| LV | 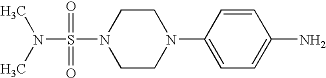 | 160 | Pinkish powder | 71 | XII |
| LVI | 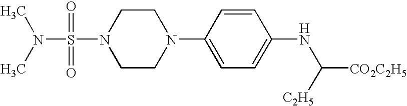 | NMR | Clear oil | 63 | I |
| LVII | 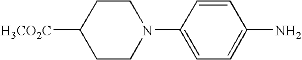 | 74 | Violet powder | 87 | XII |
| LVIII | 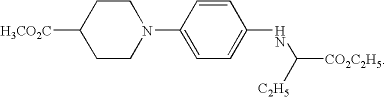 | NMR | Oil | 52 | I |
| LIX | 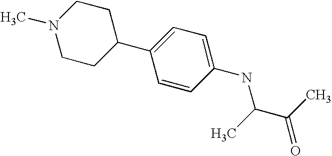 | 124 | Brown solid | 100 | VII |
| LX | 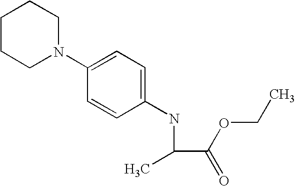 | NMR | Beige solid | 71 | Prep I |
| LXI | 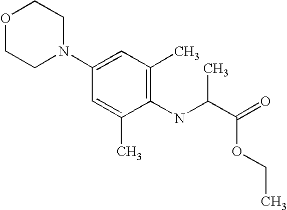 | NMR | Yellow oil | 60 | Prep I |
| LXII | 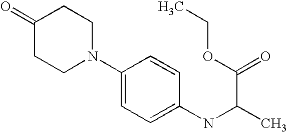 | NMR | Orange Paste | 17 | Prep I |
| LXIII | 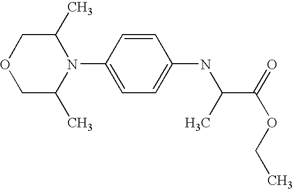 | NMR | Yellow oil | 57 | P |
| LXIV | 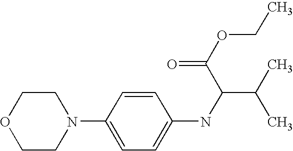 | 97 | Brown powder | 7 | Prep I |
| LXV | 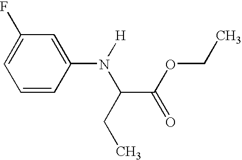 | NMR | Orange oil | 66 | Prep I |
| LXVI | 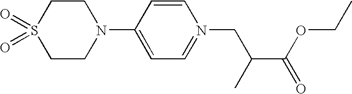 | NMR | Pink gum | 40 | Prep I |
| LXVII | 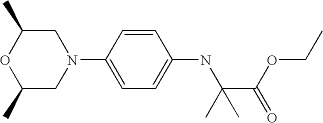 | NMR | Orange oil | 83 | Prep I |
| LXVIII | 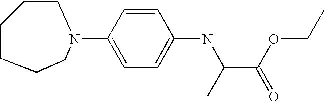 | NMR | Black oil | 66 | Prep I |
| LXIX | 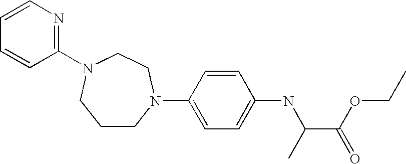 | NMR | Brown Oil | 61 | P |
| LXX | 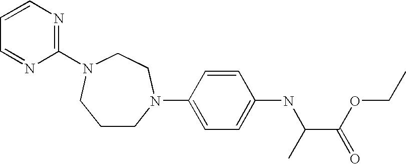 | NMR | Yellow solid | 75 | P |
| LXXI | 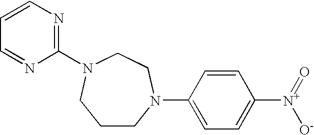 | 170 | Yellow solid | 99 | Prep XI |
| LXXII | 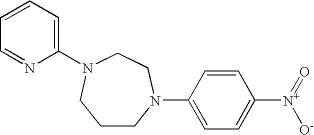 | 135 | Yellow solid | 92 | Prep XI |
| LXXIII | 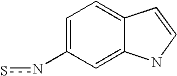 | NMR | White crystals | 30 | Prep IV |
| LXXIV | 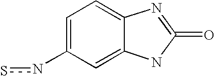 | 260 | Beige powder | 90 | Prep IV |
| LXXV | 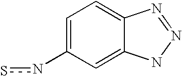 | 196 | Yellow powder | 76 | Prep IV |
| LXXVI | 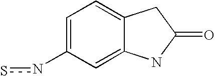 | 224 | Brown crystals | 78 | Prep IV |
| LXXVII | 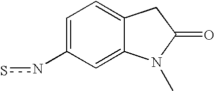 | NMR | Yellow crystals | 47 | Prep IV |
| LXXVIII | 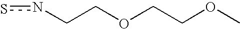 | | Not isolated | | Prep IV |

(*) Method used by analogy to that described in the preparation the number of which is indicated

TABLE III

| | | | | | M PT | Appear- | Yld | |
|---|---|---|---|---|---|---|---|---|
| Ex | R₁ | R₂ | R₃ | R₄ | ° C. | ance | % | Method |
| 51 | 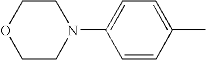 | 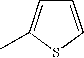 | CH₂CH₃ | H | 164 | White solid | 27 | F |
| 52 | 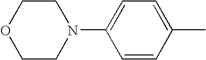 | 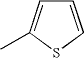 | (CH₂)₂CH₃ | H | 136 | Pinkish powder | 23 | E |
| 53 | 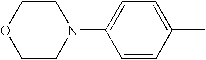 | 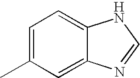 | CH₃ | H | 218-220 | Greyish powder | 75 | F |
| 54 | 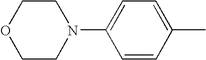 | 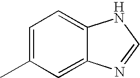 | CH₂CH₃ | H | 188-190 | White powder | 67 | F |
| 55 | 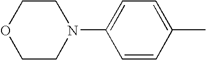 | 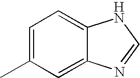 | (CH₂)₂CH₃ | OH | 264 | Greyish powder | 46 | F |
| 56 | 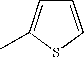 | 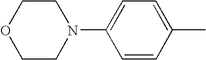 | CH₂Ch₃ | H | 222 | Greyish powder | 13 | F |
| 57 | 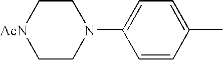 | 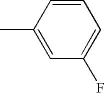 | CH₂CH₃ | H | 128 | Yellow- ish solid | 63 | F |
| 58 | 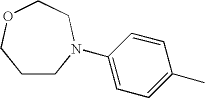 | 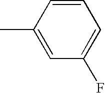 | CH₃ | H | 171 | White solid | 13 | E |
| 59 | 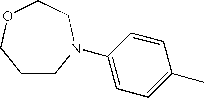 | 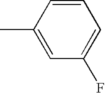 | CH₂CH₃ | H | 138 | White powder | 52 | E |
| 60 | 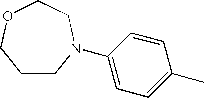 | 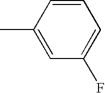 | (CH₂)₂CH₃ | H | 120 | White solid | 20 | F |
| 61 | 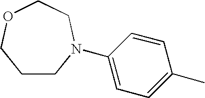 | 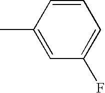 | CH₃ | CH₃ | 158 | White solid | 60 | F |
| 62 | 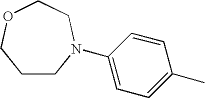 | 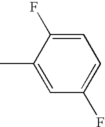 | CH₃ | H | 148 | White solid | 80 | F |
| 63 | 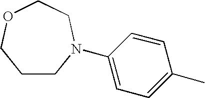 | 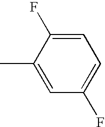 | CH₂CH₃ | H | 131 | White powder | 43 | E |
| 64 | 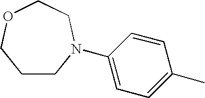 | 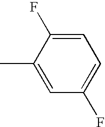 | (CH₂)₂CH₃ | H | 148 | white solid | 51 | F |
| 65 | 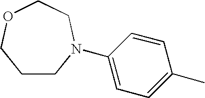 | 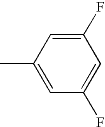 | CH₂CH₃ | H | 109 | Yellow powder | 86 | F |
| 66 | 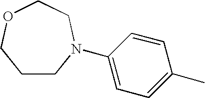 | 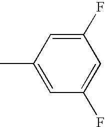 | (CH₂)₂CH₃ | H | 135-150 | Pale yellow solid | 56 | F |
| 67 | 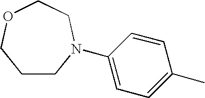 | 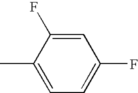 | CH₃ | H | 122 | White solid | 64 | F |
| 68 | 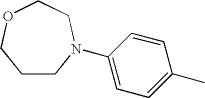 | 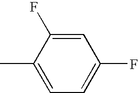 | CH₂CH₃ | H | 85-90 | Yellow foam | 65 | F |
| 69 | 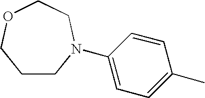 | 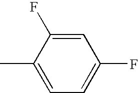 | (CH₂)₂CH₃ | H | 150 | Pale yellow solid | 49 | F |
| 70 | 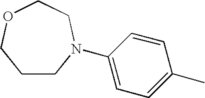 | 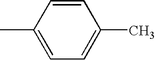 | CH₃ | H | 144 | White solid | 89 | F |
| 71 | 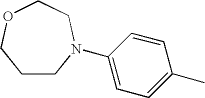 | 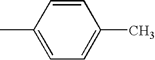 | CH₂CH₃ | H | 126 | White powder | 66 | F |
| 72 | 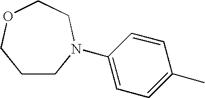 | 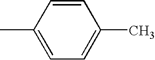 | (CH₂)₂CH₃ | H | 135 | Pale yellow solid | 25 | E |
| 73 | 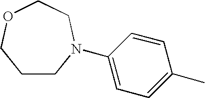 | 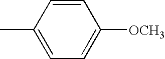 | CH₂CH₃ | H | 147 | White powder | 92 | F |
| 74 | 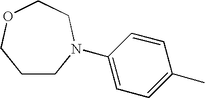 | 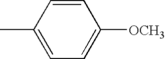 | (CH₂)₂CH₃ | H | 138 | light beige solid | 57 | F |
| 75 | 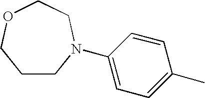 | 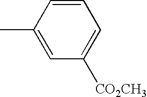 | CH₃ | H | 131 | White solid | 89 | F |
| 76 | 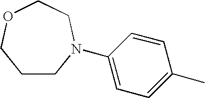 | 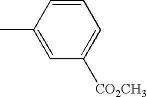 | CH₂CH₃ | H | 138 | White powder | 78 | F |
| 77 | 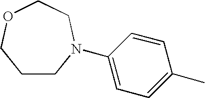 | 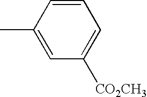 | (CH₂)₂CH₃ | H | 107 | Pink solid | 30 | F |
| 78 | 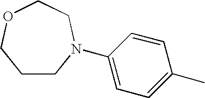 | 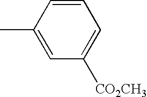 | CH₃ | CH₃ | 118 | Pink foam | 91 | F |
| 79 | 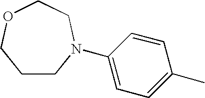 | 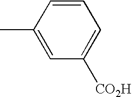 | CH₃ | H | 190 | Beige solid | 59 | F |
| 80 | 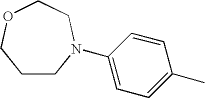 | 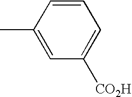 | CH₂CH₃ | H | 198 | Cream solid | 36 | F |
| 81 | 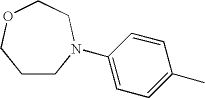 | 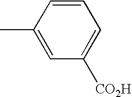 | (CH₂)₂CH₃ | H | 110-145 | Yellow glassy solid | 30 | F |
| 82 | 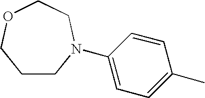 | 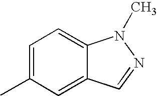 | CH₃ | H | 200-202 | White powder | 86 | F |
| 83 | 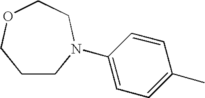 | 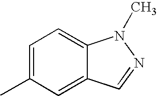 | CH₂CH₃ | H | 169-171 | White powder | 86 | F |
| 84 | 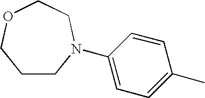 | 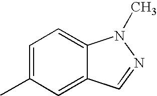 | (CH₂)₂CH₃ | H | 138-140 | White powder | 59 | F |
| 85 | 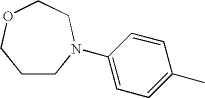 | 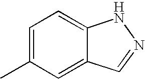 | CH₃ | H | 158-175 | Pale yellow powder | 67 | F |
| 86 | 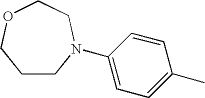 | 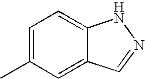 | CH₂CH₃ | H | 230-232 | light beige powder | 71 | F |
| 87 | 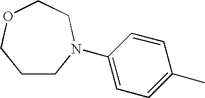 | 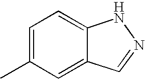 | (CH₂)₂CH₃ | H | 288-230 | Pale yellow powder | 58 | F |
| 88 | 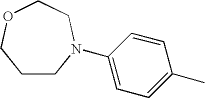 | 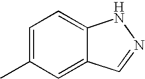 | CH₃ | CH₃ | 250 | White solid | 38 | F |
| 89 | 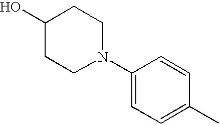 | 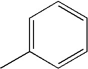 | CH₃ | H | 173 | White powder | 75 | M |
| 90 | 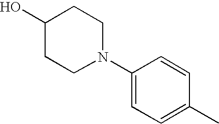 | 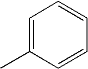 | C₂H₅ | H | 194 | White powder | 47 | M |
| 91 | 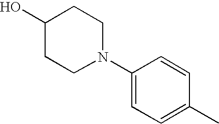 | 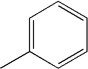 | C₃H₇ | H | 80-90 | White foam | 66 | M |
| 92 | 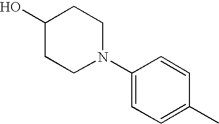 | 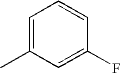 | CH₃ | H | 70 | White powder | 65 | M |
| 93 | 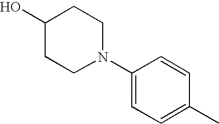 | 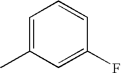 | C₂H₅ | H | 97 | Beige crystals | 30 | M |
| 94 | 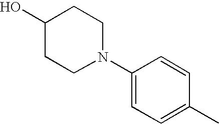 | 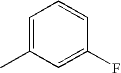 | C₃H₇ | H | 103 | Beige crystals | 54 | M |
| 95 | 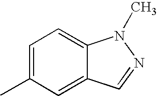 | 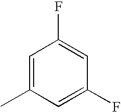 | CH₃ | H | 90-100 | White foam | 70 | M |
| 96 | 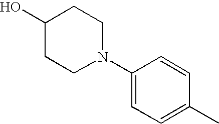 | 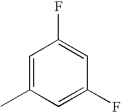 | C₂H₅ | H | 98 | White powder | 52 | M |
| 97 | 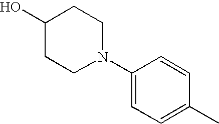 | 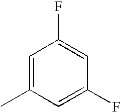 | C₃H₇ | H | 161 | White powder | 38 | M |
| 98 | 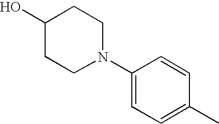 | 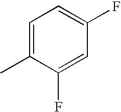 | CH₃ | H | 60 | White powder | 17 | M |
| 99 | 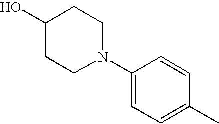 | 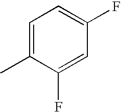 | C₂H₅ | H | 60-70 | Beige powder | 51 | M |
| 100 | 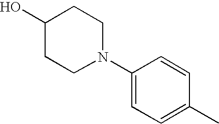 | 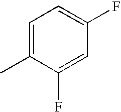 | C₃H₇ | H | 99 | White powder | 47 | M |
| 101 | 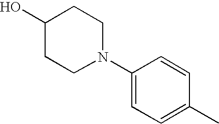 | 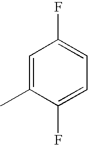 | CH₃ | CH₃ | H 60 | White powder | 67 | M |
| 102 | 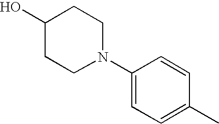 | 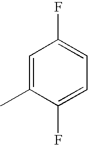 | C₂H₅ | H | 80-90 | White powder | 38 | M |
| 103 | 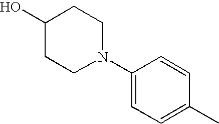 | 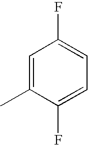 | C₃H₇ | H | 100 | Beige powder | 61 | M |
| 104 | 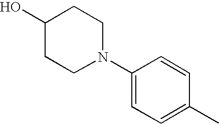 | 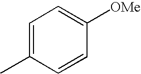 | CH₃ | H | 90 | White powder | 57 | M |
| 105 | 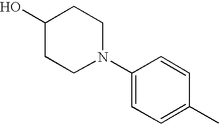 | 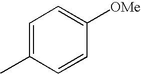 | C₂H₅ | H | 80-90 | Beige powder | 29 | M |
| 106 | 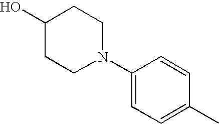 | 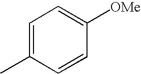 | C₃H₇ | H | 149 | White powder | 54 | M |
| 107 | 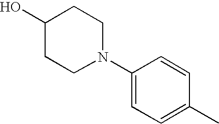 | 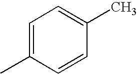 | CH₃ | H | 60 | White powder | 69 | M |
| 108 | 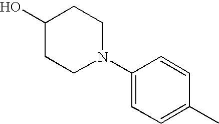 | 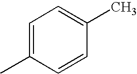 | C₂H₅ | H | 60 | White powder | 41 | M |
| 109 | 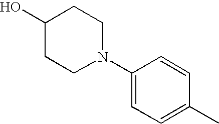 | CH3 | CH | H163 | Beige powder | 64 | M | |
| 110 | 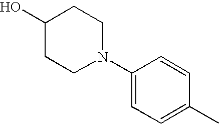 | 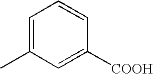 | CH₃ | H | 152 | Brown powder | 16 | M |
| 111 | 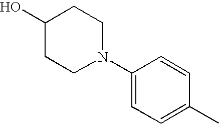 | 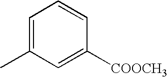 | CH₃ | H | 105 | White foam | 77 | M |
| 112 | 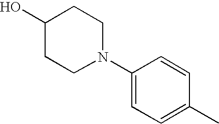 | 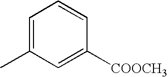 | C₂H₅ | H | 104 | Beige powder | 11 | M |
| 113 | 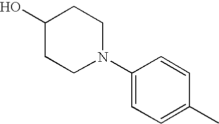 | 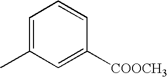 | C₃H₇ | H | 80 | Beige crystals | 37 | M |
| 114 | 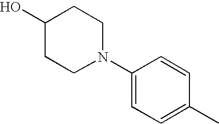 | 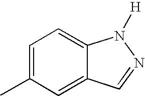 | CH₃ | H | 130-140 | White powder | 44 | M |
| 115 | 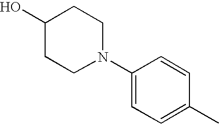 | 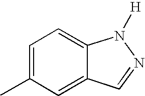 | C₂H₅ | H | 120-130 | White powder | 16 | M |
| 116 | 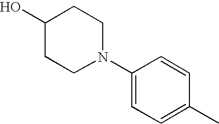 | 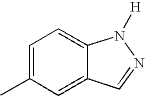 | C₃H₇ | H | 154 | White powder | 11 | M |
| 117 | 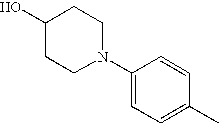 | 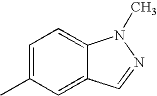 | CH₃ | H | 130 | White powder | 21 | M |
| 118 | 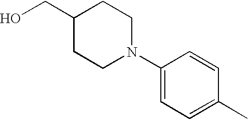 | 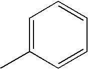 | CH₃ | H | 192-194 | White crystals | 70 | F |
| 119 | 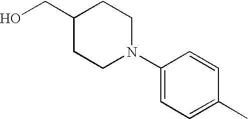 | 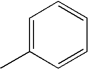 | C₂H₅ | H | 146-148 | White crystals | 66 | F |
| 120 | 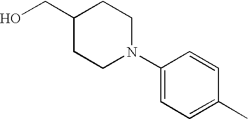 | 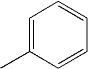 | C₃H₇ | H | 120-122 | White crystals | 55 | F |
| 121 | 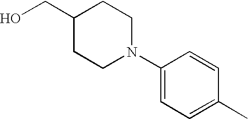 | 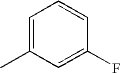 | CH₃ | H | 168-170 | White crystals | 46 | F |
| 122 | 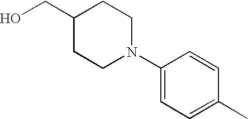 | 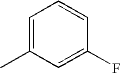 | C₂H₅ | H | 146-148 | White crystals | 53 | F |
| 123 | 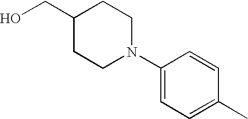 | 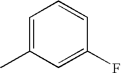 | C₃H₇ | H | 116-118 | White crystals | 51 | F |
| 124 | 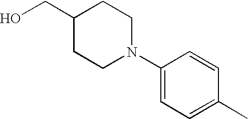 | 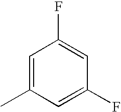 | CH₃ | H | 168-170 | White crystals | 46 | F |
| 125 | 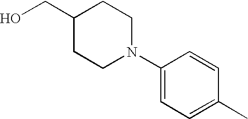 | 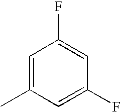 | C₁H₅ | H | 146-148 | White crystals | 52 | F |
| 126 | 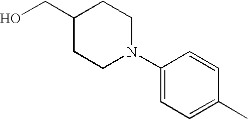 | 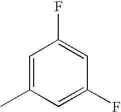 | C₃H₇ | H | 110-112 | Pale yellow crystals | 57 | F |
| 127 | 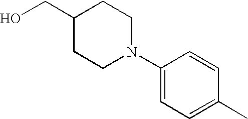 | 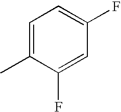 | CH₃ | H | 162-164 | White crystals | 44 | F |
| 128 | 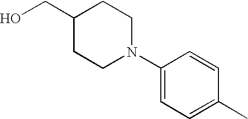 | 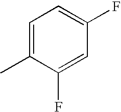 | C₂H₅ | H | 110-112 | Beige crystals | 46 | F |
| 129 | 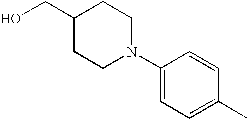 | 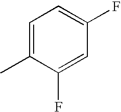 | C₃H₇ | H | 112-114 | White crystals | 23 | F |
| 130 | 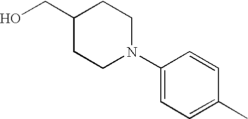 | 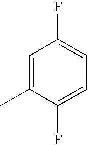 | CH₃ | H | 166-168 | White crystals | 37 | F |
| 131 | 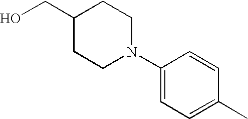 | 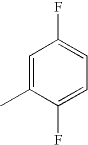 | C₂H₅ | H | 140-142 | White crystals | 63 | F |
| 132 | 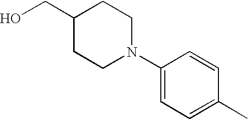 | 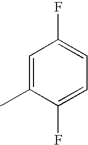 | C₃H₇ | H | 130-132 | Pale yellow crystals | 40 | F |
| 133 | 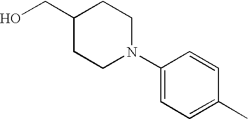 | 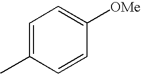 | CH₃ | H | 182-184 | White crystals | 81 | F |
| 134 | 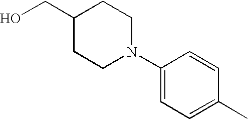 | 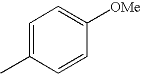 | C₂H₅ | H | 130-132 | White crystals | 66 | F |
| 135 | 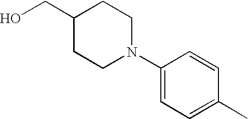 | 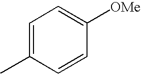 | C₃H₇ | H | 90-92 | White crystals | 44 | F |
| 136 | 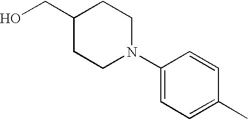 | 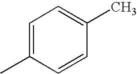 | CH₃ | H | 160-162 | White crystals | 78 | F |
| 137 | 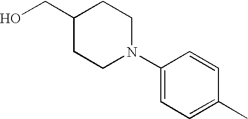 | 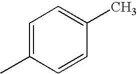 | C₂H₅ | H | 164-166 | White crystals | 57 | F |
| 138 | 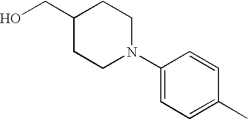 | 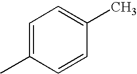 | C₃H₇ | H | 134-136 | White crystals | 49 | F |
| 139 | 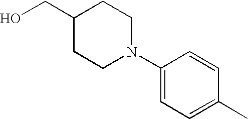 | 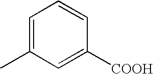 | CH₃ | H | 134-136 | Beige crystals | 20 | F |
| 140 | 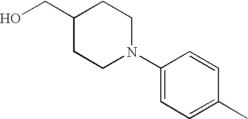 | 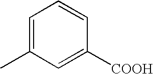 | C₂H₅ | H | 118-120 | Beige crystals | 16 | F |
| 141 | 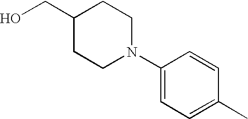 | 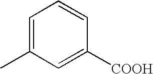 | C₃H₇ | H | 140-142 | Beige crystals | 5 | F |
| 142 | 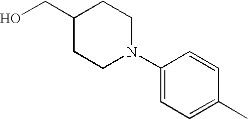 | 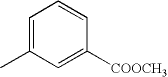 | CH₃ | H | 104-106 | White crystals | 50 | F |
| 143 | 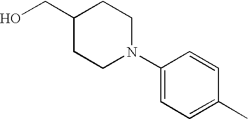 | 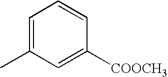 | C₂H₅ | H | 138-140 | White crystals | 50 F | |
| 144 | 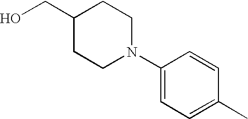 | 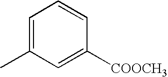 | C₃H₇ | H | 70-72 | Beige crystals | 44 | F |
| 145 | 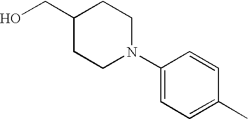 | 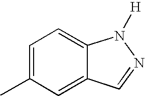 | CH₃ | H | 168-170 | White crystals | 41 | F |
| 146 | 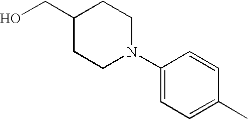 | 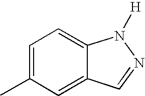 | C₂H₅ | H | 134-136 | White crystals | 55 | F |
| 147 | 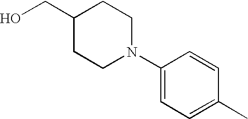 | 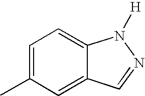 | C₃H₇ | H | 134-136 | Yellow crystals | 34 | F |
| 148 | 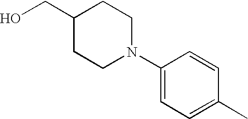 | 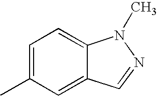 | CH₃ | H | 232-234 | Pink crystals | 16 | F |
| 149 | 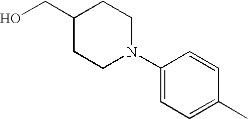 | 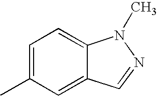 | C₂H₅ | H | 102-104 | Beige crystals | 11 | E |
| 150 | 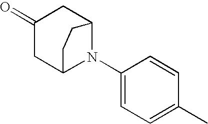 | 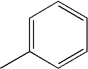 | C₂H₅ | 199 | Beige solid | 50 | F | |
| 151 | 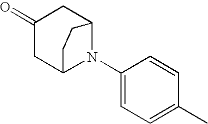 | 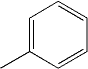 | C₃H₇ | H | 52 | Amor- phous solid | 41 | F |
| 152 | 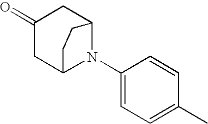 | 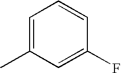 | C₂H₅ | H | 170-190 | Beige solid | 41 | F |
| 153 | 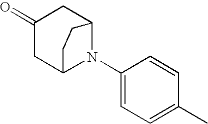 | 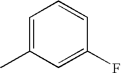 | C₃H₇ | H | 48 | Amor- phous solid | 44 | F |
| 154 | 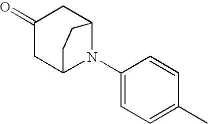 | 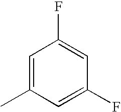 | C₂H₅ | H | 174 | Beige solid | 41 | F |
| 155 | 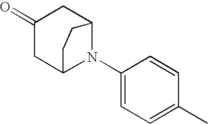 | 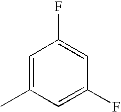 | C₃H₇ | H | 47 | Amor- phous solid | 48 | F |
| 156 | 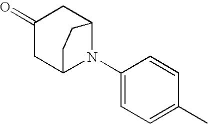 | 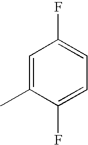 | C₂H₅ | H | 188 | Beige solid | 48 | F |
| 157 | 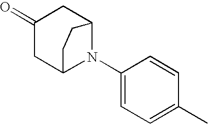 | 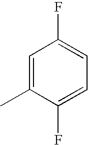 | C₃H₇ | H | 55 | Amor- phous solid | 72 | F |
| 158 | 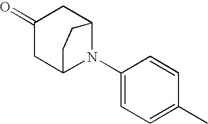 | 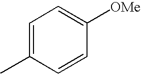 | C₃H₇ | H | 45 | Amor- phous solid | 25 | F |
| 159 | 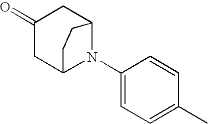 | 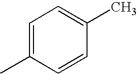 | C₂H₅ | H | 126-142 | Beige solid | 39 | F |
| 160 | 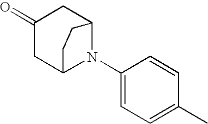 | 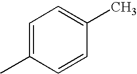 | C₃H₇ | H | 53 | Amor- phous solid | 54 | F |
| 161 | 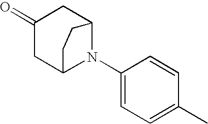 | 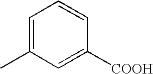 | C₃H₇ | H | 59 | Amor- phous solid | 32 | F |
| 162 | 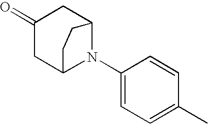 | 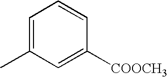 | C₂H₅ | H | 110-128 | White solid | 37 | F |
| 163 | 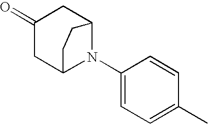 | 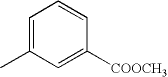 | C₃H₇ | H | 60 | Amor- phous solid | 58 | F |
| 164 | 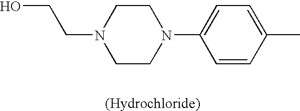 | 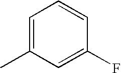 | CH₂CH₃ | H | 136-145 | Brown solid | 9 | F |
| 165 | 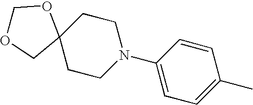 | 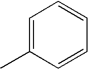 | C₂CH₅ | H | 155 | White solid | 81 | F |
| 166 | 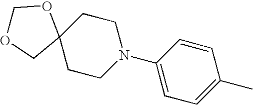 | 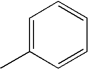 | C₃CH₇ | H | 157 | White solid | 90 | F |
| 167 | 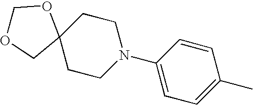 | 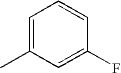 | CH₃ | H | 176 | Beige solid | 76 | F |
| 168 | 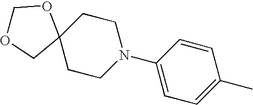 | 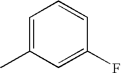 | C₂H₅ | H | 146 | Beige solid | 66 | F |
| 169 | 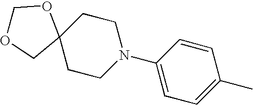 | 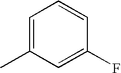 | C₃H₇ | H | 140 | Beige solid | 61 | F |
| 170 | 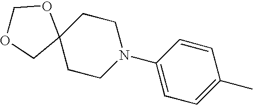 | 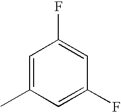 | CH₃ | H | 125 | Beige solid | 58 | F |
| 171 | 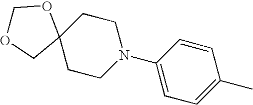 | 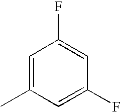 | C₂H₅ | H | 167 | light brown solid 75 | F | |
| 172 | 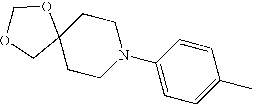 | 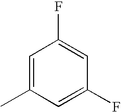 | C₃H₇ | H | 157 | Pale yellow solid | 25 | F |
| 173 | 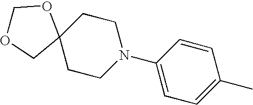 | 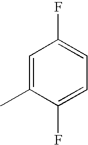 | CH₃ | H | 176 | Beige solid | 72 | F |
| 174 | 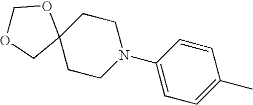 | 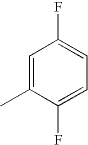 | C₂H₅ | H | 141 | Yellow solid | 42 | F |
| 175 | 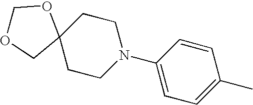 | 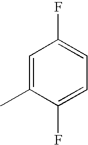 | C₃H₇ | H | 167 | Pale yellow solid | 71 | F |
| 176 | 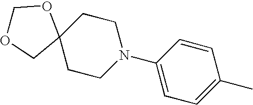 | 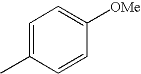 | CH₃ | H | 192 | Pale yellow solid | 90 | F |
| 177 | 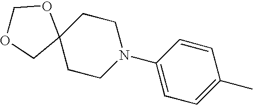 | 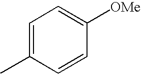 | C₂H₅ | H | 114 | Pale yellow solid | 65 | F |
| 178 | 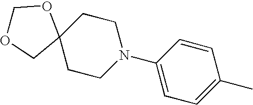 | 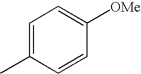 | C₃H₇ | H | 107 | White solid | 50 | F |
| 179 | 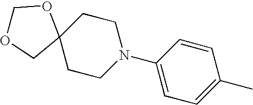 | 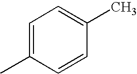 | CH₃ | H | 164 | Pale yellow solid | 76 | F |
| 180 | 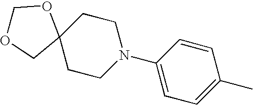 | 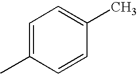 | C₂H₅ | H | 188 | Pale yellow solid | 88 | F |
| 181 | 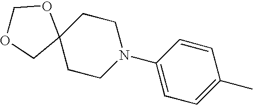 | 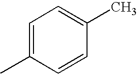 | C₃H₇ | H | 170 | White solid | 82 | F |
| 182 | 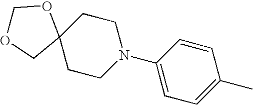 | 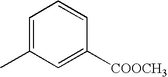 | CH₃ | H | 98 | Orange solid | 98 | F |
| 183 | 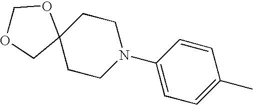 | 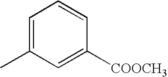 | C₂H₅ | H | 146 | light brown solid | 81 | F |
| 184 | 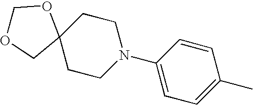 | 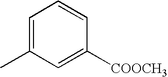 | C₃H₇ | H | 144 | Beige solid | 12 | F |
| 185 | 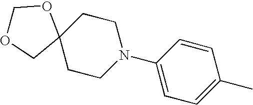 | 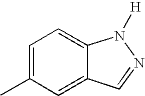 | C₃H₇ | H | 250 | White solid | 22 | F |
| 186 | 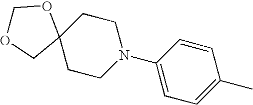 | 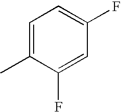 | CH₃ | H | 170 | light brown solid | 61 | F |
| 187 | 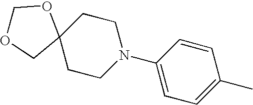 | 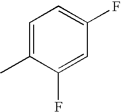 | C₂H₅ | H | 147 | Beige brown solid | 51 | F |
| 188 | 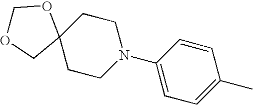 | 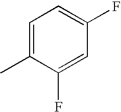 | C₃H₇ | H | 167 | White solid | 87 | F |
| 189 | 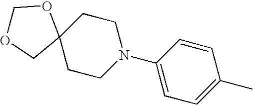 | 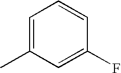 | C₂H₅ | H | 171 | White powder | 43 | F |
| 190 | 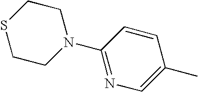 | 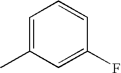 | C₂H₅ | H | 147 | White powder | 54 | F |
| 191 | 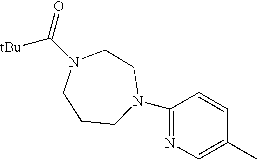 | 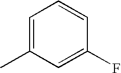 | C₂H₅ | H | 110-124 | Glassy brown solid | 60 | F |
| 192 | 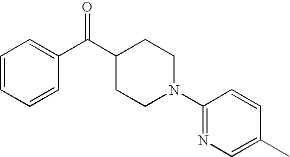 | 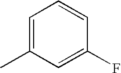 | C₂H₅ | H | 188 | Pink powder | 85 | F |
| 193 | 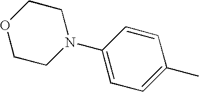 | 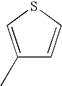 | C₂H₅ | H | 98-110 | Green- ish powder | 10 | F |
| 194 | 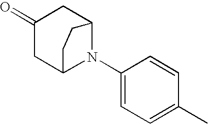 | 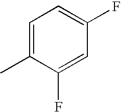 | C₂H₅ | H | 125 | Beige solid | 49 | F |
| 195 | 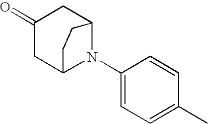 | 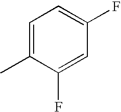 | C₃H₇ | H | 48 | Amor- phous solid | 52 | F |
| 196 | 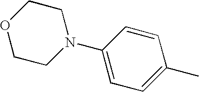 | 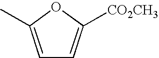 | C₂H₅ | H | 120 | Glassy yellow- ish solid | 57 | F |
| 197 | 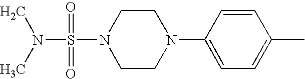 | 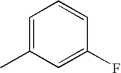 | C₂H₅ | H | 188 | White powder | 67 | F |
| 198 | 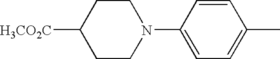 | 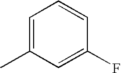 | C₂H₅ | H | 128 | Yellow powder | 51 | F |
| 199 | 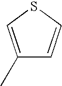 | 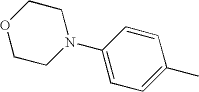 | C₂H₅ | H | 190-192 | Green- ish powder | 7 | F |
| 200 | 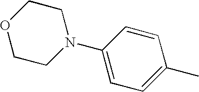 | 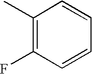 | C₂H₅ | H | 220-221 | Beige crystals | 80 | F |
| 201 | 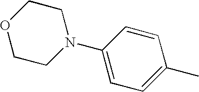 | 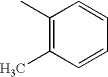 | C₂H₅ | H | 202-203 | Beige powder | 66 | F |
| 202 | 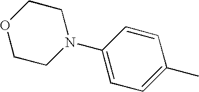 | 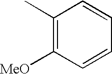 | CH₂CH₃ | H | 105 | White powder | 56 | F |
| 203 | 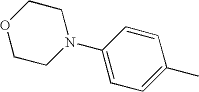 | 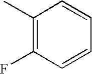 | CH₂CH₂CH₃ | H | 166 | White solid | 81 | F |
| 204 | 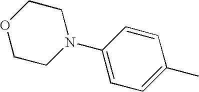 | 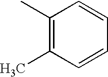 | CH₂CH₂CH₃ | H | 174 | White solid | 68 | F |
| 205 | 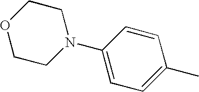 | 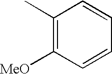 | CH₂CH₂CH₃ | H | 105 | Pale yellow powder | 92 | F |
| 206 | 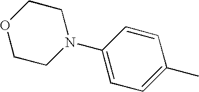 | 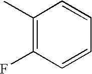 | CH₃ | CH₃ | 228 | Beige crystals | 62 | F |
| 207 | 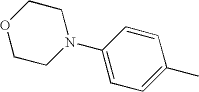 | 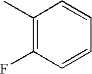 | CH₂CH₃ | H | 141-142 | Beige pinkish powder | 57 | F |
| 208 | 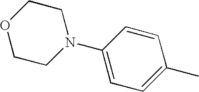 | 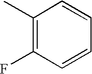 | CH₂CH₂CH₃ | H | 148 | Beige pinkish powder | 79 | F |

| Exam- | | | | | | Yld | |
|---|---|---|---|---|---|---|---|
| ple | R1 | R2 | R3 | R4 | M PT | % | Method |
| 209 | 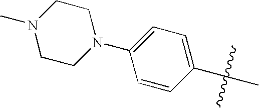 | 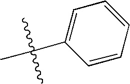 | CH3 | H | 140 | 10 | A |
| 210 | 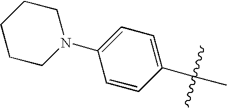 | 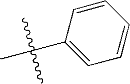 | CH3 | H | 213 | 63 | E |
| 211 | 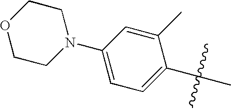 | 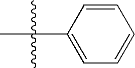 | CH2 | | 192 | 13 | E |
| 212 | 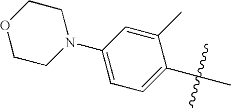 | 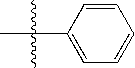 | CH3 | HO | 148 | 30 | Ex 45 |
| 213 | 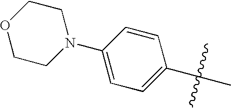 | 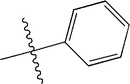 | CH3 | H | 234 | 100 | HBr SALT |
| 214 | 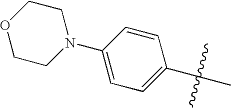 | 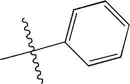 | CH3 | H | 130 | 92 | Ms SALT |
| 215 | 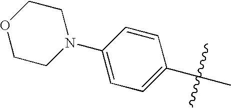 | 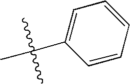 | CH3 | H | 160 | 50 | Sulph SALT |
| 216 | 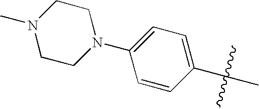 | 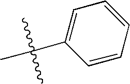 | CH3 | H | 177 | 94 | HCl SALT |
| 217 | 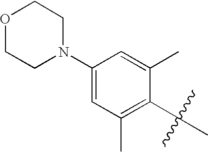 | 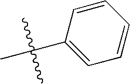 | CH3 | H | 196 | 72 | F |
| 218 | 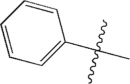 | 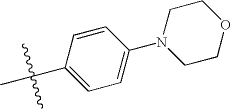 | CH3 | H | 192 | 81 | HCl SALT |
| 219 | 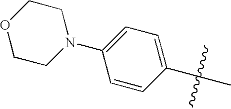 | 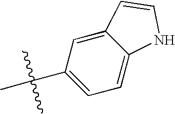 | CH3 | H | 252 | 56 | E |
| 220 | 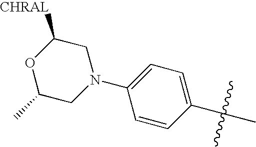 | 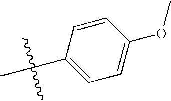 | CH3 | H | 130 | 34 | E |
| 221 | 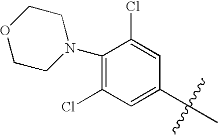 | 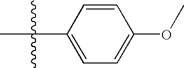 | CH3 | H | 270 | 12 | A |
| 222 | 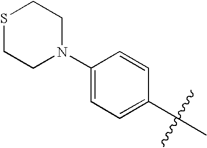 | 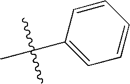 | CH3 | H | 210 | 43 | E |
| 223 | 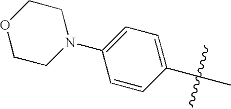 | 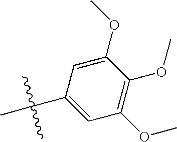 | CH3 | H | 213 | 38 | E |
| 224 | 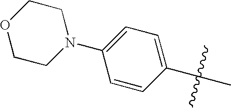 | 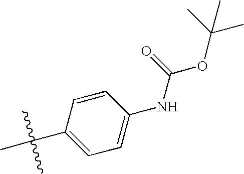 | CH3 | H | 224 | 80 | E |
| 225 | 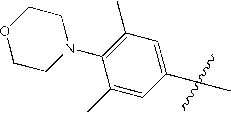 | 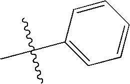 | CH3 | H | 202 | 78 | E |
| 226 | 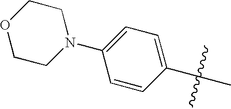 | 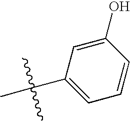 | CH3 | H | | 22 | E |
| 227 | 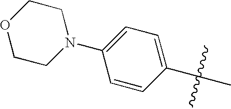 | 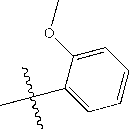 | CH3 | H | 112 | 30 | E |
| 228 | 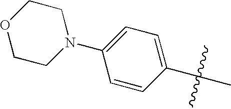 | 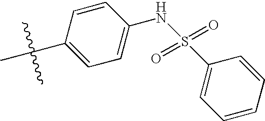 | CH3 | H | 220 | 60 | Ex 16 +RSO2Cl |
| 229 | 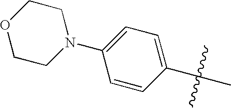 | 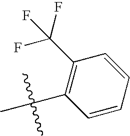 | CH3 | H | 110 | 55 | E |
| 230 | 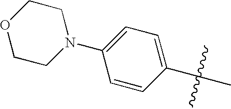 | 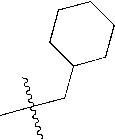 | CH3 | H | 136 | 25 | E |
| 231 | 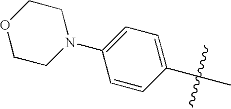 | 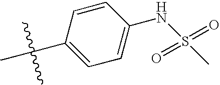 | CH3 | H | 260 | 60 | Ex 16 +RSO2Cl |
| 232 | 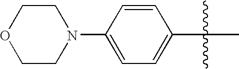 | 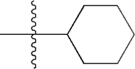 | CH3 | H | 150 | 39 | E |
| 233 | 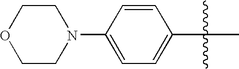 | 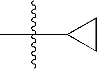 | CH3 | H | 178 | 63 | E |
| 234 | 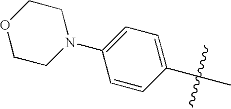 | 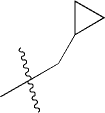 | CH3 | H | 112 | 40 | E |
| 235 | 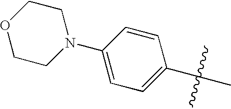 | 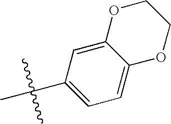 | CH3 | H | 167 | 23 | E |
| 236 | 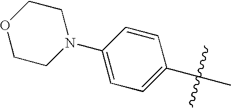 | 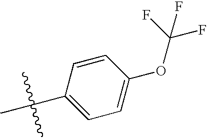 | CH3 | H | 164 | 38 | E |
| 237 | 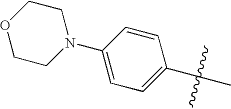 | 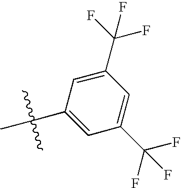 | CH3 | H | 206 | 80 | E |
| 238 | 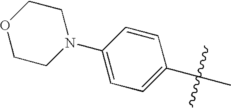 | 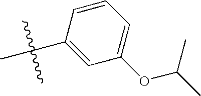 | CH3 | H | 140 | 42 | E |
| 239 | 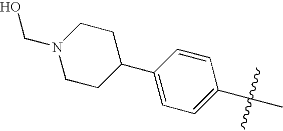 | 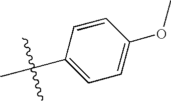 | CH3 | H | 90 | 25 | A |
| 240 | 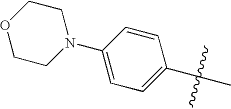 | 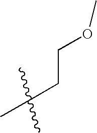 | CH3 | H | 147 | 62 | E |
| 241 | 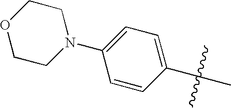 | 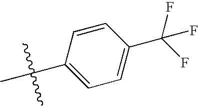 | CH3 | H | 177 | 86 | E |
| 242 | 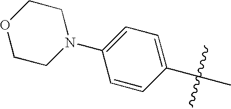 | 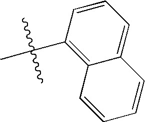 | CH3 | H | 240 | 35 | A |
| 243 | 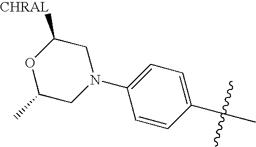 | 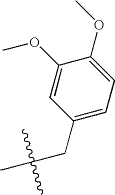 | CH3 | H | 203 | 20 | A |
| 244 | 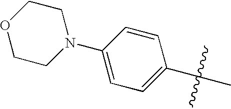 | 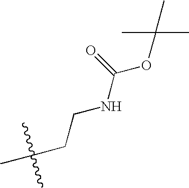 | CH3 | H | 93 | 92 | E |
| 245 | 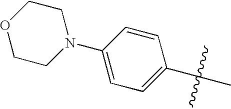 | 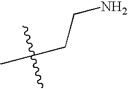 | CH3 | H | 223 | 75 | 2TFa SALT |
| 246 | 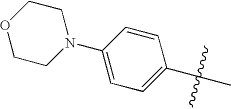 | 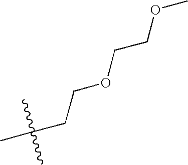 | CH3 | H | NMR | 68 | E |
| 247 | 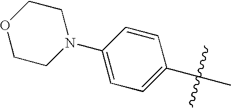 | 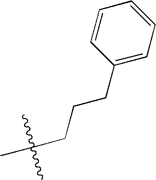 | CH3 | H | NMR | 67 | A |
| 248 | 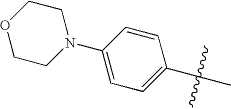 | 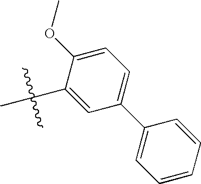 | CH3 | H | 260 | 30 | A |
| 249 | 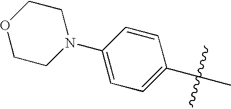 | 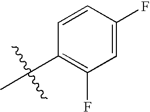 | CH3 | H | 148 | 23 | A |
| 250 | 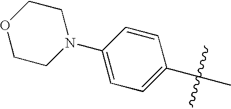 | 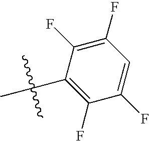 | CH3 | H | 154 | 40 | A |
| 251 | 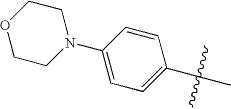 | 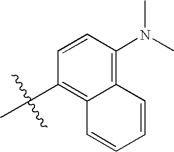 | CH3 | H | 158 | 30 | A |
| 252 | 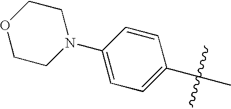 | 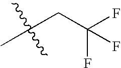 | CH3 | H | 136 | 15 | S |
| 253 | 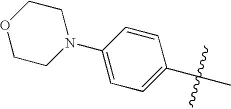 | 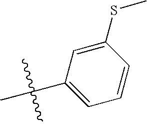 | CH3 | H | 148 | 40 | A |
| 254 | 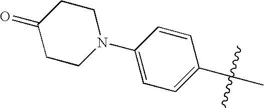 | 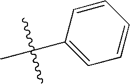 | CH3 | H | 156 | 16 | E |
| 255 | 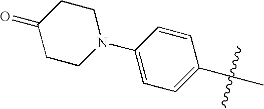 | 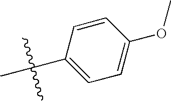 | CH3 | H | 170 | 47 | E |
| 256 | 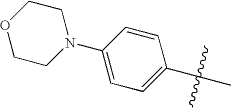 | 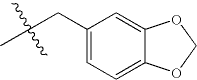 | CH3 | H | | 53 | A |
| 257 | 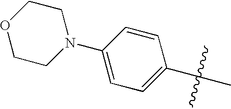 | 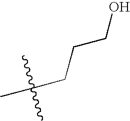 | CH3 | H | | 51 | S |
| 258 | 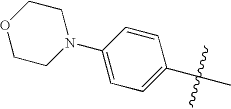 | 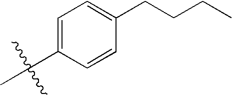 | CH3 | H | | 30 | S |
| 259 | 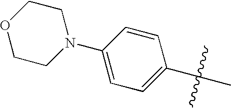 | 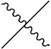 | CH3 | H | 134 | 90 | M |
| 260 | 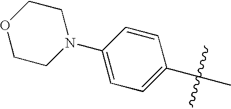 | 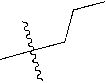 | CH3 | H | 120 | 68 | M |
| 261 | 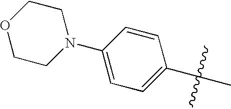 | 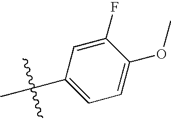 | CH3 | H | 163 | 77 | A |
| 262 | 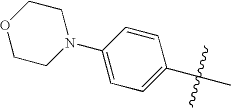 | 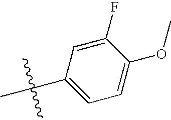 | CH3 | H | 161 | 49 | M |
| 263 | 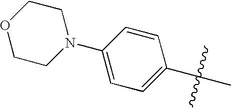 | 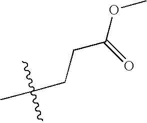 | CH3 | H | | 74 | M |
| 264 | 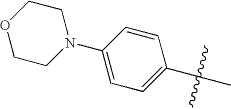 | 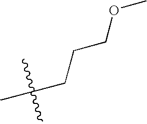 | CH3 | H | | 72 | M |
| 265 | 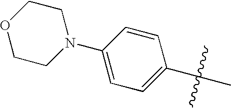 | 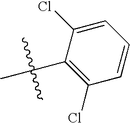 | CH3 | H | 110 | 73 | M |
| 266 | 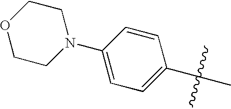 | 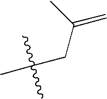 | CH3 | H | 91 | 68 | M |
| 267 | 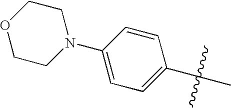 | 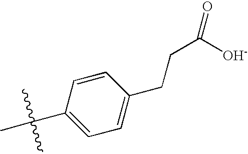 | CH3 | H | | 70 | S |
| 268 | 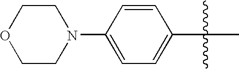 | 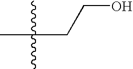 | CH3 | H | 104 | 52 | 2TFa SALT |
| 269 | 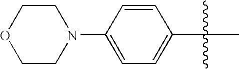 | 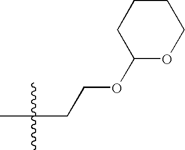 | CH3 | H | | 24 | A |
| 270 | 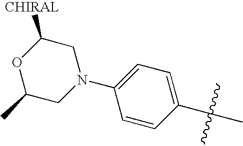 | 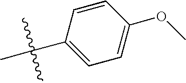 | CH3 | CH3 | 216 | 57 | E |
| 271 | 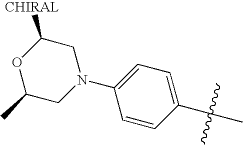 | 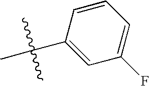 | CH3 | CH3 | 200 | 77 | E |
| 272 | 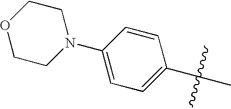 | 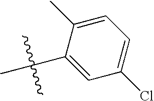 | CH3 | H | 190 | 77 | M |
| 273 | 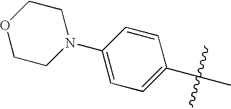 | 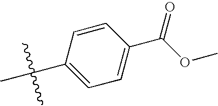 | CH3 | H | 208 | 94 | M |
| 274 | 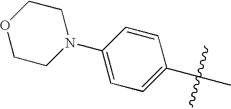 | 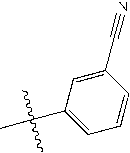 | CH3 | H | 244 | 84 | M |
| 275 | 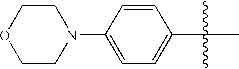 | 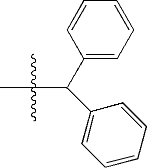 | CH3 | H | 200 | 80 | A |
| 276 | 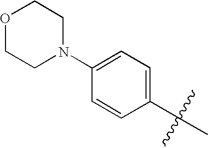 | 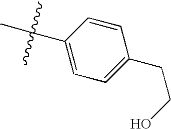 | CH3 | H | | 50 | S |
| 277 | 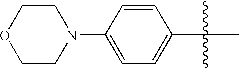 | 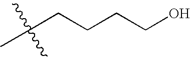 | CH3 | H | 123 | 25 | S |
| 278 | 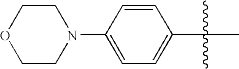 | 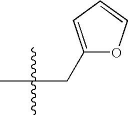 | CH3 | H | 161 | 58 | M |
| 279 | 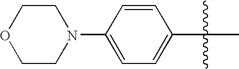 | 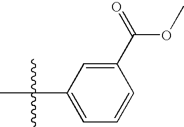 | CH3 | H | 140 | 80 | M |
| 280 | 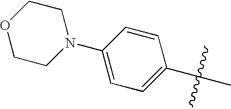 | 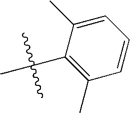 | CH3 | H | 193 | 78 | M |
| 281 | 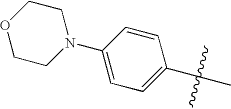 | 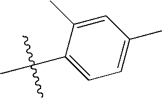 | CH3 | H | 172 | 92 | M |
| 282 | 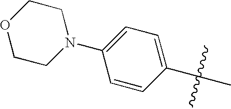 | 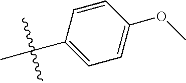 | C2H5 | H | 96 | 50 | E |
| 283 | 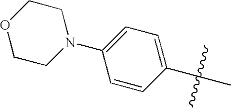 | 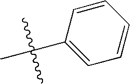 | C2H5 | H | 194 | 57 | E |
| 284 | 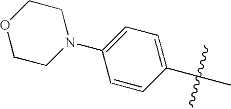 | 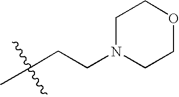 | CH3 | H | 70 | 61 | E |
| 285 | 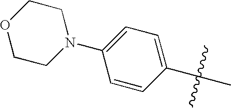 | 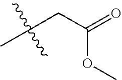 | CH3 | H | | 92 | E |
| 286 | 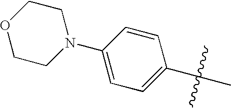 | 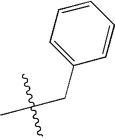 | CH3 | H | 84 | 83 | M |
| 287 | 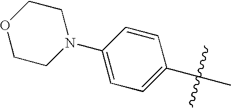 | 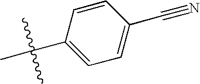 | CH3 | H | 254 | 88 | M |
| 288 | 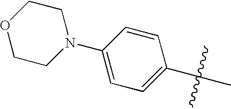 | 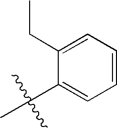 | CH3 | H | 148 | 83 | M |
| 289 | 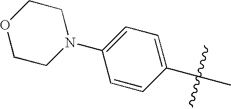 | 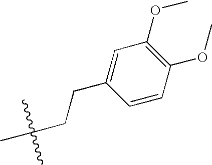 | CH3 | H | 154 | 80 | E |
| 290 | 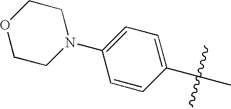 | 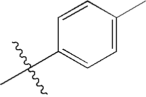 | CH3 | H | 183 | 46 | E |
| 291 | 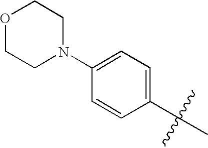 | 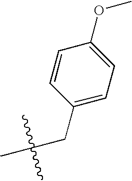 | CH3 | H | 90 | 69 | E |
| 292 | 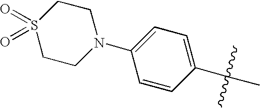 | 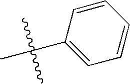 | CH3 | H | 140 | 18 | E |
| 293 | 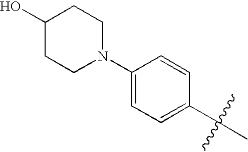 | 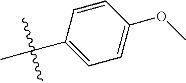 | CH3 | H | 92 | 57 | M |
| 294 | 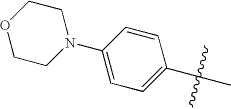 | 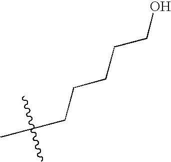 | CH3 | H | | 12 | S |
| 295 | 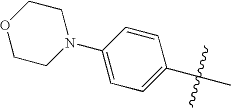 | 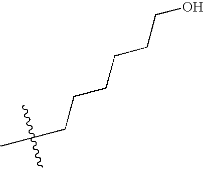 | CH3 | H | | 15 | S |
| 296 | 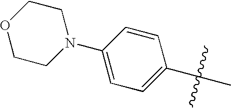 | 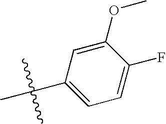 | CH3 | H | 211 | 73 | A |
| 297 | 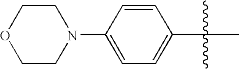 | 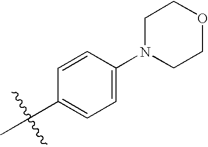 | CH3 | H | 140 | 44 | A |
| 298 | 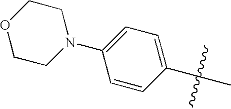 | 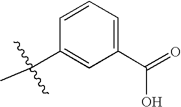 | CH3 | H | 260 | 86 | E |
| 299 | 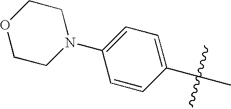 | 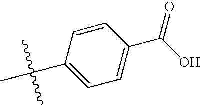 | CH3 | H | 242 | 80 | E |
| 300 | 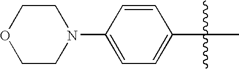 | 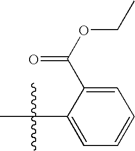 | CH3 | H | 105 | 71 | M |
| 301 | 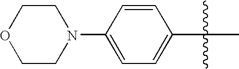 | 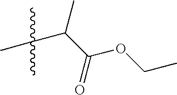 | CH3 | H | | 90 | A |
| 302 | 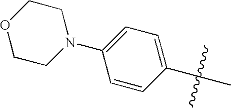 | 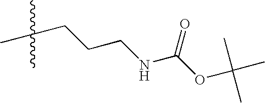 | CH3 | H | | 68 | E |
| 303 | 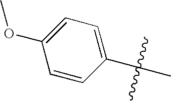 | 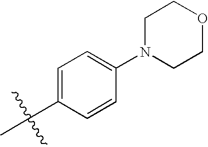 | CH3 | H | 203 | 54 | S |
| 304 | 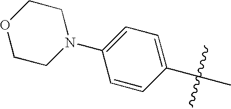 | 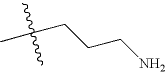 | CH3 | H | 180 | 73 | 2TFa SALT |
| 305 | 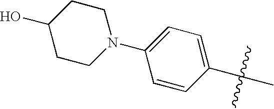 | 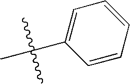 | CH3 | H | 190 | 75 | M |
| 306 | 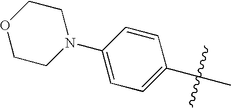 | 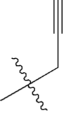 | CH3 | H | 144 | 22 | A |
| 307 | 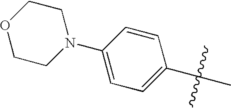 | 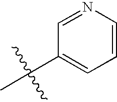 | CH3 | H | 120 | 94 | 2HCl SALT |
| 308 | 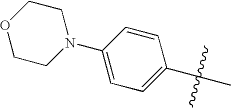 | 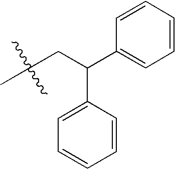 | CH3 | H | 90 | 64 | M |
| 309 | 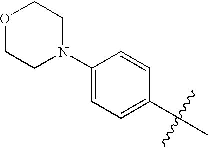 | 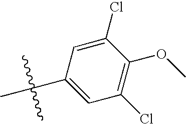 | CH3 | H | 180 | 29 | A |
| 310 | 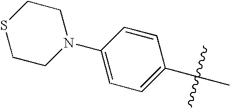 | 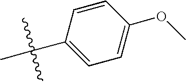 | CH3 | H | 173 | 12 | E |
| 311 | 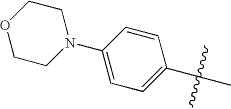 | 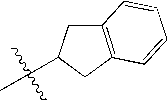 | CH3 | H | 183 | 71 | M |
| 312 | 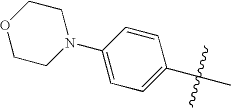 | 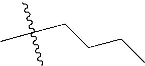 | CH3 | H | | 58 | M |
| 313 | 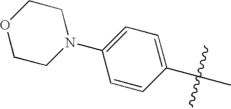 | 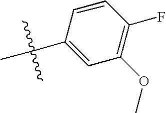 | CH3 | H | 190 | 21 | M |
| 314 | 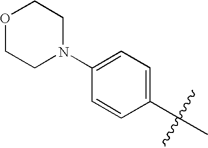 | 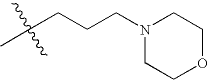 | CH3 | H | | 32 | A |
| 315 | 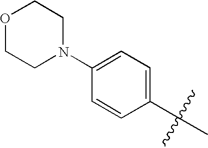 | 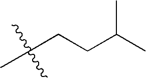 | CH3 | H | | 76 | A |
| 316 | 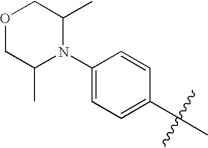 | 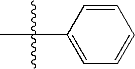 | CH3 | H | 208 | 72 | E |
| 317 | 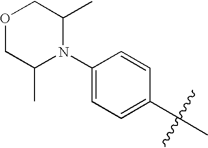 | 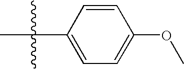 | CH3 | H | 214 | 58 | E |
| 318 | 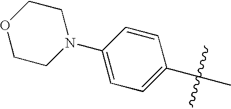 | 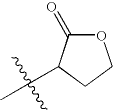 | CH3 | H | 170 | 68 | M |
| 319 | 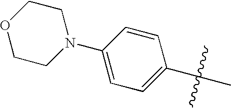 | 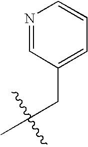 | CH3 | H | 95 | 62 | M |
| 320 | 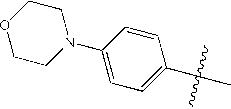 | 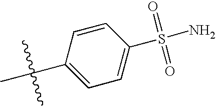 | CH3 | H | 195 | 75 | M |
| 321 | 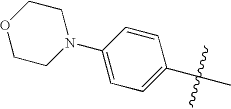 | 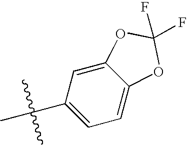 | CH3 | H | 174 | 22 | S |
| 322 | 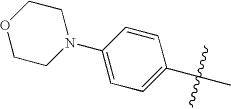 | 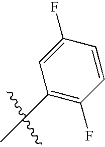 | CH3 | H | 145 | 50 | A |
| 323 | 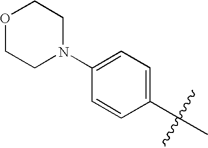 | 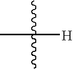 | CH3 | H | 220 | 56 | E |
| 324 | 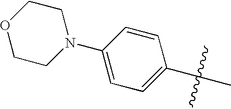 | 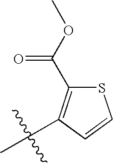 | CH3 | H | 88 | 23 | M |
| 325 | 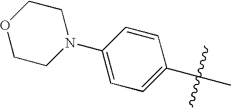 | 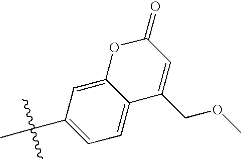 | CH3 | H | | 6 | E |
| 326 | 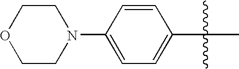 | 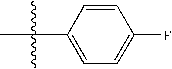 | CH3 | CH3 | 188 | 63 | M |
| 327 | 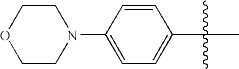 | 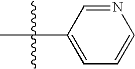 | CH3 | CH3 | 183 | 66 | M |
| 328 | 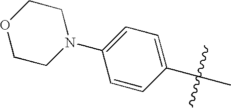 | 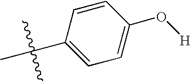 | CH3 | CH3 | 290 | 32 | M |
| 329 | 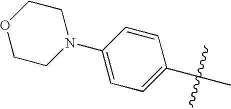 | 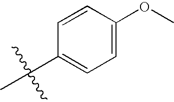 | HO | CH3 | 240 | 10 | Ex 45 |
| 330 | 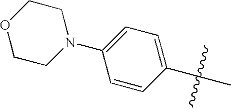 | 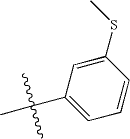 | CH3 | CH3 | 204 | 73 | M |
| 331 | 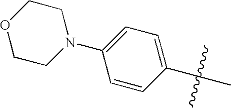 | 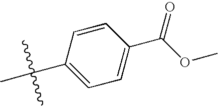 | CH3 | CH3 | 260 | 80 | M |
| 332 | 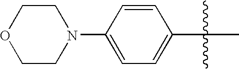 | 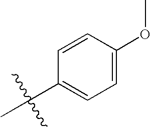 | CH2 | | 224 | 9 | Ex 1 +dmso reflux +air 8h |
| 333 | 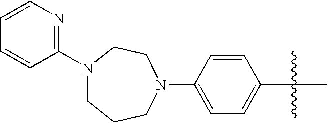 | 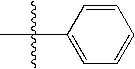 | CH3 | H | 180 | 44 | M |
| 334 | 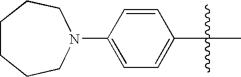 | 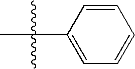 | CH3 | H | 168 | 83 | M |
| 335 | 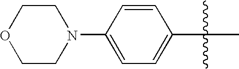 | 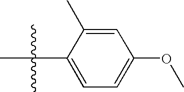 | CH3 | CH3 | 178 | 40 | M |
| 336 | 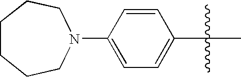 | 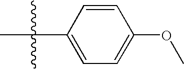 | CH3 | H | 191 | 74 | M |
| 337 | 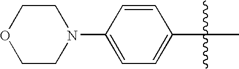 | 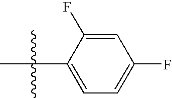 | CH3 | CH3 | 170 | 51 | M |
| 338 | 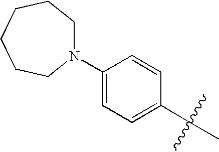 | 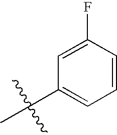 | CH3 | H | 140 | 82 | M |
| 339 | 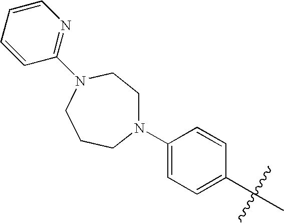 | 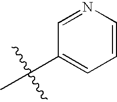 | CH3 | H | 206 | 61 | M |
| 340 | 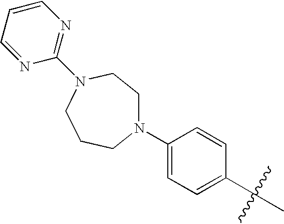 | 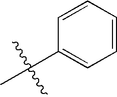 | CH3 | H | 221 | 80 | M |
| 341 | 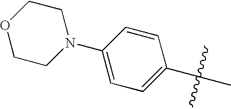 | 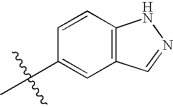 | CH3 | CH3 | 170 | 14 | A |
| 342 | 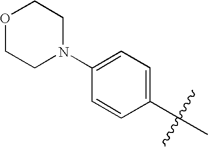 | 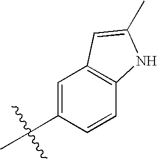 | CH3 | CH3 | 260 | 13 | S |
| 343 | 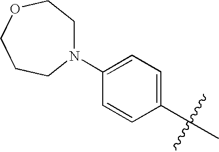 | 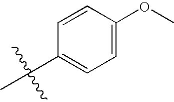 | CH3 | H | 158 | 59 | M |
| 344 | 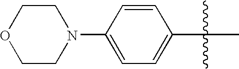 | 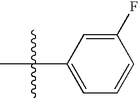 | C2H5 | H | 161 | 12 | M |
| 345 | 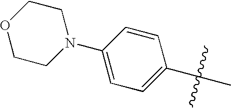 | 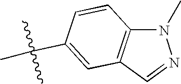 | CH3 | CH3 | 228 | 50 | A |
| 346 | 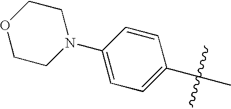 | 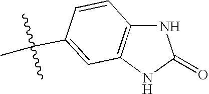 | CH3 | CH3 | 174 | 17 | A |
| 347 | 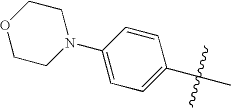 | 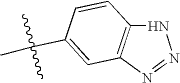 | CH3 | CH3 | 260 | 81 | A |
| 348 | 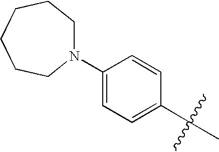 | 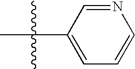 | CH3 | H | 85 | 79 | M |
| 349 | 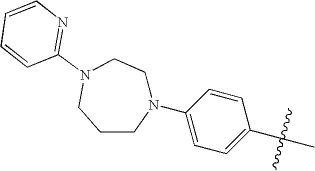 | 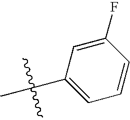 | CH3 | H | 150 | 45 | M |
| 350 | 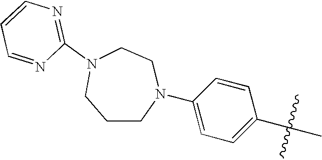 | 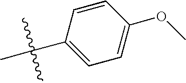 | CH3 | H | 217 | 76 | M |
| 351 | 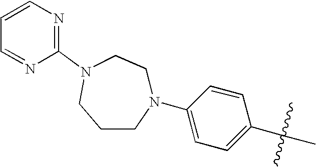 | 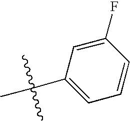 | CH3 | H | 196 | 75 | M |
| 352 | 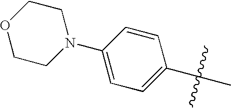 | 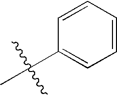 | C3H7 | H | 90 | 79 | M |
| 353 | 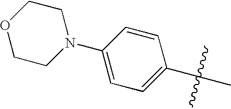 | 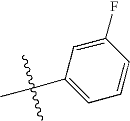 | C3H7 | H | 90 | 93 | M |
| 354 | 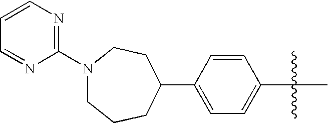 | 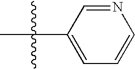 | CH3 | H | 241 | 66 | M |
| 355 | 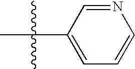 | 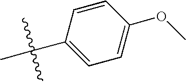 | CH3 | H | 192 | 54 | M |
| 356 | 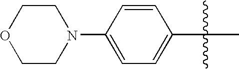 | 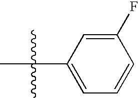 | C3H7 | H | 60 | 52 | M |
| 357 | 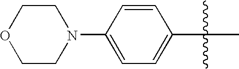 | 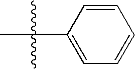 | C3H7 | H | 179 | 46 | M |
| 358 | 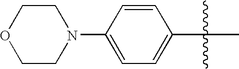 | 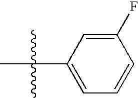 | HO | CH3 | 100 | 44 | Ex 45 |
| 359 | 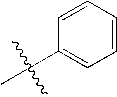 | 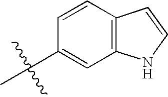 | CH3 | CH3 | 144 | 12 | S |
| 360 | 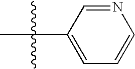 | 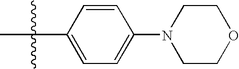 | C2H5 | H | 189 | 64 | A |
| 361 | 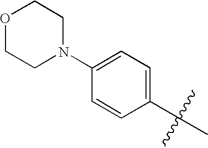 | 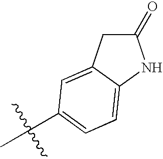 | CH3 | CH3 | 172 | 14 | M |
| 362 | 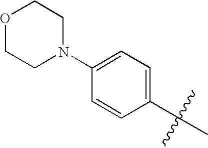 | 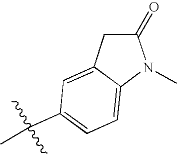 | CH3 | CH3 | 158 | 12 | M |
| 363 | 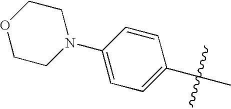 | 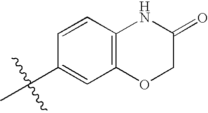 | CH3 | CH3 | 260 | 100 | A |
| 364 | 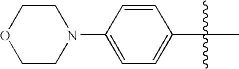 | 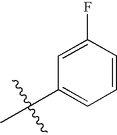 | CH3 | H | 150 | 78 | F |
| 365 | 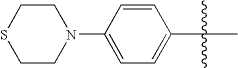 | 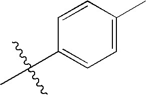 | CH3 | H | 186 | 50 | M |
| 366 | 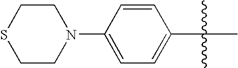 | 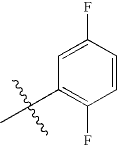 | CH3 | H | 88 | 98 | F |
| 367 | 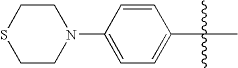 | 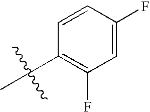 | CH3 | H | 176 | 70 | F |
| 368 | 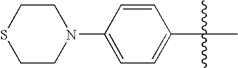 | 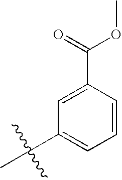 | CH3 | H | 98 | 87 | F |
| 369 | 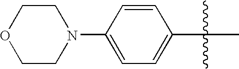 | 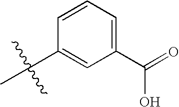 | CH3 | H | 250 | 93 | F |
| 370 | 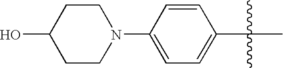 | 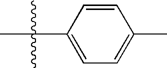 | CH3 | H | 60 | 69 | M |
| 371 | 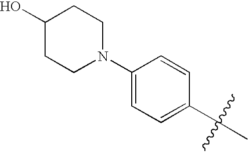 | 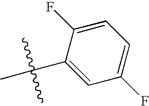 | CH3 | H | 60 | 67 | M |
| 372 | 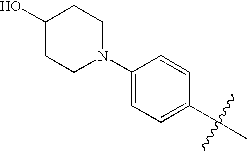 | 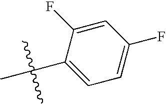 | CH3 | H | 60 | 17 | M |
| 373 | 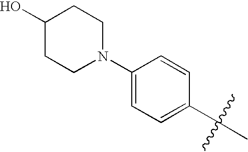 | 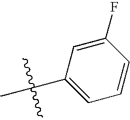 | CH3 | H | 70 | 65 | M |
| 374 | 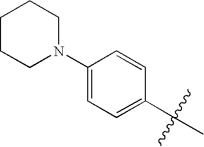 | 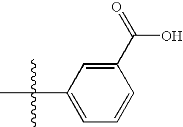 | CH3 | H | 258 | 83 | A |
| 375 | 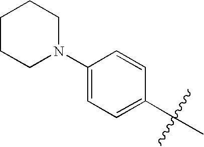 | 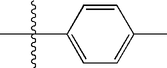 | CH3 | H | 176 | 74 | A |
| 376 | 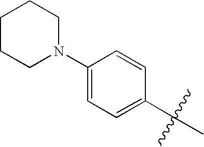 | 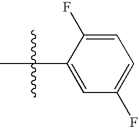 | CH3 | H | 150 | 98 | A |
| 377 | 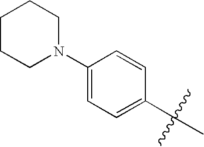 | 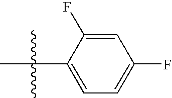 | CH3 | H | 156 | 37 | A |
| 378 | 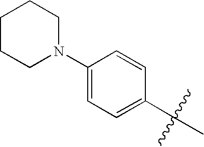 | 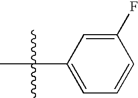 | CH3 | H | 144 | 38 | A |
| 379 | 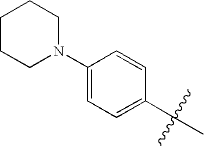 | 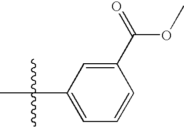 | CH3 | H | | 88 | A |
| 380 | 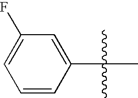 | 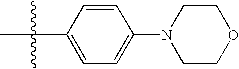 | C2H5 | H | 182 | 71 | F |

The non-crystallised compounds appearing in the Tables above were characterised by their proton NMR spectrum, the values of which (chemical shift, form and intensity of signal), are given below:

Preparation XXI

¹H NMR (DMSO d₆, 300 MHz): 1.86 (m, 2H); 3.39 (m, 4H); 3.55 (m, 2H); 3.66 (m, 2H); 4.30 (s, 2H); 6.50 (m, 4H).

Preparation XXII

¹H NMR (DMSO d₆, 250 MHz): 1.15 (t, 3H); 1.32 (d, 3H); 1.86 (m, 2H); 3.41 (m, 4H); 3.55 (m, 2H); 3.67 (m, 2H); 3.91 (m, 1H); 4.06 (q, 2H); 5.16 (d, 1H); 6.47 (m, 2H); 6.56 (m, 2H).

Preparation XXIII

¹H NMR (CDCl₃, 250 MHz): 1.00 (t, 3H); 1.24 (t, 3H); 1.81 (m, 2H); 2.00 (m, 2H); 3.51 (m, 4H); 3.70 (m, 3H); 3.79 (m, 2H); 3.89 (m, 1H); 4.18 (q, 2H); 6.61 (m, 4H).

Preparation XXIV

¹H NMR (DMSO d₆, 300 MHz): 0.89 (t, 3H); 1.14 (t, 3H); 1.40 (m, 2H); 1.66 (m, 2H); 1.87 (m, 2H); 3.40 (m, 4H); 3.55 (m, 2H); 3.66 (m, 2H); 3.82 (m, 1H); 4.06 (q, 2H); 5.13 (d, 1H); 6.48 (m, 2H); 6.55 (m, 2H).

Preparation XXVII

¹H NMR(CDCl₃, 250 MHz): 1.22 (t, 3H); 1.45 (s, 6H); 2.00 (m, 2H); 3.53 (m, 4H); 3.68 (m, 3H); 3.80 (m, 2H); 4.15 (q, 2H); 6.58 (m, 2H); 6.70 (m, 2H).

Preparation XXVIII

¹H NMR (CDCl₃, 300 MHz): 1.00 (t, 3H); 1.24 (t, 3H); 1.80 (m, 2H); 2.61 (t, 2H); 2.68 (m, 4H); 3.07 (m, 4H); 3.66 (t, 2H); 3.92 (m, 2H); 4.17 (q, 2H); 6.60 (m, 2H); 6.82 (m, 2H).

Preparation XXXI

¹H NMR (DMSO d₆, 300 MHz): 0.94 (t, 3H); 1.14 (t, 3H); 1.48 (m, 2H); 1.73 (m, 4H); 2.59 (m, 2H); 3.24 (m, 1H); 3.50 (m, 1H); 3.80 (m, 1H); 4.40 (q, 2H); 4.60 (s, 1H); 5.37 (d, 1H); 6.49 (d, 2H); 6.71 (d, 2H).

Preparation XXXII

¹H NMR (DMSO d₆, 300 MHz): 0.89 (t, 3H); 1.16 (t, 3H); 1.43 (m, 4H); 1.69 (m, 4H); 2.54 (m, 2H); 3.24 (m, 2H); 3.52 (m, 1H); 3.84 (m, 1H); 4.37 (q, 2H); 4.60 (s, 1H); 5.37 (d, 1H); 6.46 (d, 2H); 6.71 (d, 2H).

Preparation XLI

¹H NMR (DMSO d₆, 250 MHz) 0.89 (t, 3H); 1.17 (t,3H); 1.39 (m, 2H); 1.59 (m, 6H); 3.00 (t, 4H); 3.82 (m, 5H); 4.06 (q, 2H); 7.40 (d, 1H); 6.47 (d, 2H); 6.74 (d,2H).

Preparation XLIV

¹H NMR (CDCl₃, 300 MHz): 2.67 (m, 4H); 3.27 (s, 2H); 3.77 (m, 4H); 6.54 (d, 1H); 6.98 (dd, 1H); 7.78 (d, 1H).

Preparation XLV

¹H NMR (CDCl₃, 250 MHz): 1.01 (t, 3H); 1.25 (t, 3H); 1.82 (m, 2H); 2.68 (m, 4H); 3.76 (m, 5H); 3.86 (m, 1H); 4.18 (m, 2H); 6.55 (d, 1H); 6.95 (dd, 1H); 7.73 (d, 1H).

Preparation XLVI

¹H NMR (CDCl₃, 300 MHz): 1.23 (m, 9H); 1.96 (m, 2H); 3.46 (m, 2H); 3.60 (m, 2H); 3.76 (m, 4H); 6.42 (d, 1H); 6.97 (dd, 1H); 7.73 (d,2H).

Preparation XLVII

¹H NMR (CDCl₃, 300 MHz): 1.01 (t, 3H); 1.22 (s, 9H); 1.27 (t, 3H); 1.80 (m, 2H); 1.96 (m, 2H); 3.45 (m, 2H); 3.60 (m, 3H); 3.80 (m, 4H); 4.17 (m, 2H); 6.44 (d, 1H); 6.95 (dd, 1H); 7.68 (d, 1H).

Preparation XLVIII

¹H NMR (CDCl₃, 300 MHz): 1.00 (t, 3H); 1.25 (t, 3H); 1.81 to 1.95 (m, 6H); 2.91 (m, 2H); 3.43 (m, 1H); 3.73 (m, 1H); 3.88 (m, 1H); 4.16 (m, 4H); 6.64 (d, 1H); 6.96 (dd, 1H); 7.45 to 7.59 (m, 3H); 7.74 (d, 1H); 7.96 (m, 2H).

Preparation LIV

¹H NMR (CDCl₃, 300 MHz): 3.89 (s, 3H); 6.17 (d, 1H); 7.14 (d, 1H).

Preparation LVI

¹H NMR (CDCl₃, 300 MHz): 1.0 (t, 3H); 1.24 (t, 3H); 1.82 (m, 2H); 2.85 (s, 6H). 3.07 (m, 4H); 3.39 (m, 4H); 3.94 (m, 2H); 4.17 (q, 2H); 6.60 (d, 2H); 6.82 (d, 2H).

Preparation LVIII

¹H NMR (CDCl₃, 300 MHz): 1.0 (t, 3H); 1.24 (t, 3H); 1.73 to 2.01 (m, 6H); 2.40 (m, 1H); 2.67 (m, 2H); 3.42 (m, 2H); 3.70 (s, 3H); 3.93 (m, 2H); 4.17 (q, 2H); 6.59 (d, 2H); 6.85 (m, 2H).

Preparation LX

¹H NMR (CDCl₃, 300 MHz): 1.24 (t, 3H); 1.44 (d, 3H); 1.52 (m, 2H); 1.70 (m, 4H); 2.98 (m, 4H); 4.05 (q, 1H); 4.15 (q, 2H); 6.58 (d, 2H); 6.86 (d, 2H).

Preparation LXI

¹H NMR (CDCl₃, 300 MHz): 1.20 (t, 3H); 1.32 (d, 3H); 2.22 (s, 3H); 2.26 (s, 3H); 3.82 (m, 2H); 3.96 (m, 2H); 3.85 (m, 4H); 4.11 (q, 2H); 4.54 (q, 1H) 6.71 (d, 2H).

Preparation LXII

¹H NMR (CDCl₃, 300 MHz): 1.27 (t, 3H); 1.45 (d, 3H); 2.56 (t, 4H); 3.42 (t, 4H); 4.08 (q, 1H); 4.20 (q, 2H); 6.62 (d, 2H); 6.89 (d, 2H).

Preparation LXIII

¹H NMR (CDCl₃, 300 MHz) 1.23 (d, 6H); 1.24 (t, 3H); 1.44 (d, 3H); 2.32 (d, 2H); 3.25 (d, 2H); 3.82 (m, 2H); 4.08 (q, 1H); 4.16 (q, 2H); 6.61 (d, 2H) 6.85 (d, 2H).

Preparation LXV

¹H NMR (CDCl₃, 300 MHz): 1.01 (t, 3H); 1.28 (t, 3H); 1.81 (m, 2H); 3.98 (m, 1H); 4.21 (q, 2H); 6.37 (m, 3H); 7.08 (q, 1H).

Preparation LXVI

¹H NMR (CDCl₃, 300 MHz): 1.26 (t, 3H); 1.46 (d, 3H); 3.13 (m, 4H); 3.62 (m, 4H); 4.07 (q, 1H); 4.21 (q, 2H); 6.59 (d, 2H); 6.82 (d, 2H).

Preparation LXVII

¹H NMR (CDCl₃, 300 MHz) 1.17 (t, 3H); 1.30 (d, 6H); 1.49 (s, 6H); 2.34 (m, 2H); 3.28 (d, 2H); 3.80 (m, 2H); 4.14 (q, 2H); 6.64 (d, 2H); 6.77 (d, 2H).

Preparation LXVIII

¹H NMR (CDCl₃, 250 MHz): 1.28 (t, 3H); 1.44 (d, 3H); 1.53 (m, 4H); 1.81 (m, 4H); 3.41 (m, 4H); 4.17 (m, 3H); 6.53 (m, 4H).

Preparation LXIX

¹H NMR (DMSO d₆, 250 MHz): 1.13 (t, 3H); 1.32 (d, 3H); 1.93 (m, 2H); 3.28 (t, 4H); 3.45 (t, 4H); 3.77 (m, 2H); 3.90 (m, 1H); 4.05 (q, 2H); 5.15 (d, 1H) 6.52 (m, 6H); 7.43 (m, 1H); 8.03 (m, 1H).

Preparation LXX

¹H NMR (CDCl₃, 250 MHz) 1.23 (t, 3H); 1.43 (d, 3H); 2.06 (q, 2H); 3.43 (t, 2H); 3.56 (t, 2H); 3.67 (t, 2H); 3.98 (m, 3H); 4.16 (q, 2H); 6.44 (t, 1H); 6.62 (q, 4H); 8.17 (d, 2H).

Preparation LXXIII

¹H NMR (CDCl₃, 250 MHz): 6.56 (m, 1H); 7.10 (d, 1H); 7.28 (m, 1H); 7.38 (d, 1H); 7.55 (d, 1H); 8.27 (s, 1H).

Preparation LXXVII

¹H NMR (DMSO d₆, 250 MHz): 3.11 (s, 3H); 3.57 (s, 2H); 7.02 (d, 1H); 7.37 (m, 2H).

### EXAMPLE 226

¹H NMR(CDCl₃, 300 MHz): 1.51 (d, 3H); 3.22 (s, 4H); 3.86 (s, 4H); 4.58 (q, 1H); 5.41 (s, 1H); 6.90 (m, 4H); 7.32 (m, 4H).

### EXAMPLE 246

¹H NMR (CDCl₃, 250 MHz): 1.39 (d, 3H); 3.21 (q, 4H); 3.37 (s, 3H); 3.53 (m, 2H); 3.68 (m, 2H); 3.84 (m, 6H); 4.12 (m, 2H); 4.37 (q, 1H); 6.95 (d, 2H); 7.21 (d, 2H).

### EXAMPLE 247

¹H NMR (CDCl₃, 250 MHz): 1.32 (d, 3H); 2.09 (m, 2H); 2.72 (t, 2H); 3.21 (q, 2H); 3.86 (m, 4H); 3.97 (t, 2H); 4.23 (q, 1H) 6.94 (m, 2H); 7.21 (m, 7H).

### EXAMPLE 256

¹H NMR (CDCl₃, 250 MHz): 1.38 (d, 3H); 3.20 (q, 4H); 3.84 (q, 4H); 4.36 (q, 1H); 5.01 (q, 2H); 5.93 (s, 2H); 6.75 (d, 1H); 6.92 (m,2H); 7.05 (m, 2H); 7.22 (m, 2H).

### EXAMPLE 257

¹H NMR (CDCl₃, 250 MHz): 1.41 (d, 3H); 1.94 (m, 2H); 2.73 (s, 1H); 3.22 (m, 2H); 3.62 (s, 2H); 3.85 (m, 4H); 4.09 (t, 2H); 4.40 (q, 1H); 6.95 (m, 2H) 7.24 (m, 2H).

### EXAMPLE 258

¹H NMR (CDCl₃, 250 MHz): 0.93 (m, 3H); 1.37 (m, 2H); 1.51 (d, 3H); 1.65 (m, 2H); 2.66 (m, 2H); 3.22 (m, 4H); 3.85 (m, 4H); 4.58 (q, 1H); 6.96 (m, 2H) 7.28 (m, 6H).

### EXAMPLE 263

¹H NMR (CDCl₃, 300 MHz): 1.40 (d, 3H); 2.79 (2t, 2H); 3.21 (t, 4H); 3.69 (s, 3H); 3.86 (t, 4H); 4.21 (t, 2H); 4.38 (q, 1H); 6.95 (d, 2H); 7.23 (d, 2H).

### EXAMPLE 264

¹H NMR (CDCl₃, 300 MHz) 1.39 (d, 3H) 2.02 (m, 2H); 3.21 (m, 4H); 3.32 (s, 3H); 3.48 (t, 2H); 3.86 (m, 4H); 4.01 (t, 2H); 4.35 (q, 1H) 6.95 (d, 2H); 7.24 (d, 2H).

### EXAMPLE 267

¹H NMR (DMSO d₆, 300 MHz): 1.33 (d, 3H); 2.58 (t, 2H); 2.86 (t, 2H); 3.16 (m, 2H); 3.44 (s, 1H); 3.74 (m, 2H); 4.97 (q, 1H); 7.03 (d, 2H); 7.27 (d, 2H); 7.36 (m, 4H)

### EXAMPLE 269

¹H NMR (CDCl₃, 300 MHz): 1.40 (d, 3H); 1.61 (m, 6H); 3.22 (m, 4H); 3.51 (m, 1H); 3.82 (m, 2H); 3.86 (t, 4H); 4.06 (m, 2H); 4.23 (m, 1H); 4.39 (q, 1H); 4.74 (t, 1H) 6.96 (d, 2H); 7.24 (d, 2H).

### EXAMPLE 276

¹H NMR (CDCl₃, 250 MHz): 1.54 (d, 3H); 2.92 (t, 2H); 3.22 (m, 4H); 3.88 (m, 6H); 4.58 (q, 1H); 6.97 (m, 2H); 7.31 (m, 6H).

### EXAMPLE 285

¹H NMR (CDCl₃, 300 MHz): 1.44 (t, 3H); 3.21 (m, 4H); 3.79 (s, 3H); 3.86 (m, 4H); 4.48 (m, 4H); 4.48 (q, 1H); 4.69 (d, 2H); 6.94 (d, 2H); 7.26 (d, 2H).

### EXAMPLE 294

¹H NMR (CDCl₃, 250 MHz): 1.30 (m, 2H); 1.37 (d, 3H); 1.75 (m, 2H); 1.81 (m, 2H); 3.21 (m, 4H); 3.66 (t, 2H); 3.85 (m, 4H); 3.92 (m, 2H); 4.36 (q, 1H); 6.96 (d, 2H); 7.24 (d, 2H).

### EXAMPLE 295

¹H NMR (CDCl₃, 300 MHz): 1.35 (d, 3H); 1.45 (m, 4H); 1.61 (m, 2H); 1.80 (m, 2H); 3.22 (q, 4H); 3.65 (t, 2H); 3.85 (m, 2H); 3.90 (m, 2H); 4.35 (q, 1H); 6.97 (m, 2H); 7.24 (m, 2H).

### EXAMPLE 301

¹H NMR (CDCl₃, 300 MHz) 1.27 (m, 3H); 1.41 (m, 3H); 1.69 (d, 3H); 3.21 (m, 4H); 3.86 (m, 4H); 4.23 (m, 2H); 4.38 (t, 1H); 5.50 (m, 1H); 6.95 (d, 2H); 7.25 (m, 2H).

### EXAMPLE 302

¹H NMR (CDCl₃, 300 MHz): 1.41 (m, 12H); 1.90 (t, 2H); 3.20 (m, 4H); 3.86 (m, 4H); 3.99 (t, 2H); 4.38 (q, 1H); 5.1 (m, 1H); 6.94 (d, 2H); 7.23 (d, 2H).

### EXAMPLE 312

¹H NMR (CDCl₃, 250 MHz): 0.97 (t, 3H); 1.39 (m, 5H); 1.70 (m, 2H); 3.21 (m, 4H); 3.88 (m, 6H); 4.35 (q, 1H); 6.95 (d, 2H); 7.24 (d, 2H).

### EXAMPLE 314

¹H NMR (CDCl₃, 250 MHz): 1.39 (d, 3H); 1.92 (m, 2H); 2.43 (m, 6H); 3.21 (m, 4H); 3.71 (t, 4H); 3.86 (q, 4H); 4.36 (q, 1H); 6.95 (m, 2H); 7.24 (m, 2H).

### EXAMPLE 315

¹H NMR (CDCl₃, 300 MHz): 0.98 (d, 6H); 1.38 (d, 3H); 1.61 (m, 2H); 3.21 (m, 4H); 3.88 (m, 6H); 4.34 (q, 1H); 6.94 (d, 2H); 7.24 (m, 2H).

### EXAMPLE 325

¹H NMR (CDCl₃, 300 MHz) 1.54 (d, 3H); 3.23 (t, 4H); 3.51 (s, 3H); 3.86 (m, 4H); 4.6 (d, 2H); 6.59 (d, 1H); 6.98 (m, 2H); 7.21 (m, 1H); 7.32 (m, 4H).

### EXAMPLE 379

¹H NMR (CDCl₃, 300 MHz): 1.53 (d, 3H); 1.69 (m, 6H); 3.23 (m, 4H); 3.92 (s, 3H); 4.59 (q, 1H); 6.97 (d, 2H); 7.24 (d, 1H); 7.25 (m, 2H); 7.59 (m, 2H); 8.08 (m, 2H).

Compounds of formula I according to the invention were subjected to pharmacological tests in order to evaluate their potential to decrease the level of glycaemia in the blood.

Experimental Method

In vivo studies were made on male C57BL/KsJ-db/db mice, originating from CERI] (Route des Chênes Secs—BP 5—53940 Le Genest St Isle—France).

The animals were accommodated in cages equipped with a filter cover and have free access to an irradiated standard nourishment as well as to filtered drinking water. All the material used (cages, feeding bottles, pipettes and shavings) is sterilised by autoclave, irradiation or soaking in a disinfectant. The temperature of the room is kept at 23±2° C. The light and dark cycle is of 12 hours.

During the acclimatisation period, each animal is labelled with the aid of an electronic chip, the implantation of which is done under anaesthetic by inhalation of a CO₂/O₂ mixture.

Groups of 10 mice are made and the treatments start when the animals are 10 to 11 weeks old. The products are placed in suspension in gum arabic at 3% and are administered to the animals with the aid of a feeding cannula, for 10 days, at the rate of two administrations per day, as well as the morning of the eleventh day. The products are tested of doses of less than 200 mg/kg. The animals of the control group receive the administration vehicle only.

A blood sample is taken before treatment, and then three hours after the last administration of the product. The animals are anaesthetised by inhalation of a CO₂/O₂ mixture, the blood is taken from the retro-orbital sinus, collected in a dry tube and kept in the cold. The serum is prepared by centrifugation at 2,800 g (15 minutes, 4° C.) in the hour following the sampling. The samples are kept at −20° C. until the analysis.

The serum levels of glucose and triglycerides are determined with a Konelab 30 analyser, with the aid of Konelab kits. The animals the glycaemia of which before treatment was less than 3 g/l are systematically excluded from the study.

For each group, the average levels of glucose and triglycerides before and after treatment are calculated and the results are expressed in percentage variation of these averages with time.

The results expressed in percentage variation of the level of glycaemia and of the level of triglycerides show that the compounds of formula I according to the invention or their addition salts with a non-toxic acid, enable the level of glycaemia to drop to values of −73% and the level of triglycerides to values of −56%. It was also observed that the treatment with the compounds according to the invention were accompanied with a favourable modification of the lipid parameters.

The compounds according to the invention can be used as an active principle of a medicament which is intended for treating diabetes in mammals and, more particularly, in man. They can be used for fighting against hypertriglyceridaemiae and diseases caused by an excess of triglycerides in the blood, such as atherosclerosis, for example.

More generally, the compounds can be useful for the prevention or the treatment of diseases associated with a hyperglycaemia or a hypertriglyceridaemia, such as adult diabetes, hypertension, dyslipidaemiae, cardiovascular diseases, and obesity; they are also useful for the treatment of diseases caused by microvascular or macrovascular complications in the diabetic, notably of the renal system or the central nervous system, said complications being in general associated with the X metabolic syndrome. The compounds according to the invention are also useful for treating cerebral ischaemia or cerebral vascular accident.

Pharmaceutical compositions incorporating the compounds according to the invention can be formulated notably by combination of these compounds with usual non-toxic excipients, according to methods which are well-known to the person skilled in the art, preferably so as to obtain medicaments which may be administered via the oral route, e.g. capsules or tablets. Practically, in case of administration of the compound via the oral route, the daily dosage in man will preferably be between 5 and 500 mg. Although the formulations in the form of capsules or tablets be preferred for reasons of comfort of the patient, the compounds according to the invention can also be prescribed in other galenic forms, e.g. if the patient does not accept or is not in a state to accept the solid oral formulations, or if the treatment necessitates a very rapid bioavailability of the active principle. It will therefore be possible for the medicament to be presented in the form of a drinkable syrup, or in injectable form, preferably sub-cutaneous or intramuscular.

1. A thiohydantoin derivative compound, characterised in that it is selected from:
a) compounds of formula 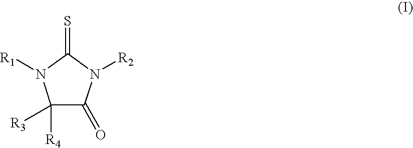
in which
R₁ represents an aromatic ring which is non-substituted or substituted with one or more atoms or groups of atoms selected from halogens, linear or branched C₁-C₄ alkoxy, linear, branched or cyclic C₁-C₄ alkyl, linear or branched C₁-C₄ alkylthio, nitro, trifluoromethyl, trifluoromethoxy, methylenedioxy, or 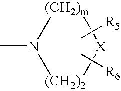
groups,
R₂ represents:
a hydrogen atom,
a linear, branched or cyclic C₁-C₇ alkyl group, optionally having one or more oxygen atoms,
a C₁-C₃ haloalkyl group,
a linear or branched C₃-C₅ alkenyl group,
a linear or branched C₃-C₄ alkynyl group,
a C₂-C₆ hydroxyalkyl group,
a C₂-C₄ aminoalkyl group,
a C₂-C₃ cyanoalkyl group,
a linear or branched C₁-C₃ alkyl group, which is substituted with one or more R₇ substituents, or
an aromatic ring which is non-substituted or substituted with one or more atoms or groups of atoms selected from halogens, linear or branched C₁-C₄ alkoxy, linear, branched or cyclic C₁-C₄ alkyl, linear or branched C₁-C₄ alkylthio, amino, cyano, hydroxy, nitro, trifluoromethyl, trifluoromethoxy, methylenedioxy, ethylenedioxy, difluoromethylenedioxy, aminosulphonyl, dimethylamino, C₁-C₃ hydroxyalkyl, carboxylic acid, C₂-C₃ alkyl ester, methanesulphonylamino, benzenesulphonylamino, t-butoxycarbonylamino, or 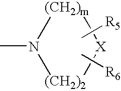
groups,
R₃, R₅ and R₆ each independently represent a hydrogen atom or a C₁-C₄ alkyl group,
R₄ represents a hydrogen atom, a C₁-C₄ alkyl group or a hydroxy group, or,
R₃ and R₄ together form a methylene group, or
R₅ and R₆ together form an ethylene group —CH₂—CH₂—,
R₇ represents a carboxylic acid group which is free or esterified with a C₁-C₃ alkyl group, a phenyl ring which is non-substituted or substituted with one or more methoxy, phenyl or methylenedioxy groups, a 2-furyl ring, a 2-, 3- or 4-pyridinyl ring or a 4-morpholinyl group,
m=2 or 3,
X represents an oxygen atom, a sulphur atom, a sulphoxide group, a sulphonyl group, a carbonyl group, a 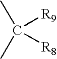
group, or a: 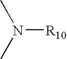
group,
R₈ represents a hydrogen atom, a hydroxy group, a C₁-C₂ hydroxyalkyl group, a benzoyl group or a CO₂CH₃ group,
R₉ represents a hydrogen atom or forms, with R₈, an ethylenedioxy group, and
R₁₀ represents a methyl group, a C₂-C₄ hydroxyalkyl group, a 1-oxo-C₂-C₄-alkyl group, an SO₂N(CH₃)₂ group, a 2-pyridinyl group or a 2-pyrimidinyl group,
on the condition that at least one of the R₁ and R₂ substituents represents an aromatic ring which is substituted at least with a 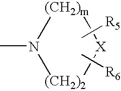
group,
and
b) addition salts of the compounds of formula I with an acid, notably pharmaceutically acceptable salts.

2. The compound according to claim 1, characterised in that it is selected from:
a) compounds of formula 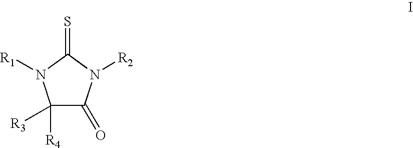
in which
R₁ represents a phenyl ring which is optionally substituted with one or more atoms or groups of atoms selected from halogens, linear C₁-C₄ alkyl or 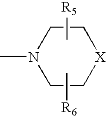
groups,
R₂ represents
a linear or cyclic C₁-C₇ alkyl group,
a linear C₃-C₅ alkenyl group, or
a phenyl, 2-thienyl or 3-pyridinyl ring, which is optionally substituted with one or more atoms or groups of atoms selected from halogens, linear or branched C₁-C₄ alkoxy, linear C₁-C₄ alkyl, linear C₁-C₄ alkylthio, amino, hydroxy, nitro, trifluoromethyl, trifluoromethoxy, methylenedioxy or 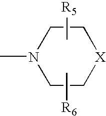
groups,
R₃ represents a hydrogen atom, a linear C₁-C₄ alkyl group, or a hydroxy group,
R₄, R₅, and R₆ each independently represent a hydrogen atom or a linear C₁-C₄ alkyl group,
X represents an oxygen atom, a sulphoxide group or a carbon atom which is substituted with a C₁-C₂ hydroxyalkyl group,
on the condition that at least one of the R₁ and R₂ substituents represents an aromatic ring which is substituted at least with a 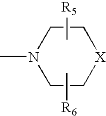
group,
and
b) addition salts of compounds of formula I with an acid, notably pharmaceutically acceptable salts.

3. The compound according to claim 1, characterised in that R₁ represents a phenyl group which is substituted in the para position with a 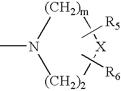
group,
in which X, m, R₅ and R₆ are as defined in claim 1.

4. The compound according to one of claims 1 to 3, characterised in that X represents an oxygen atom.

5. The compound according to one of claims 1 to 4, characterised in that R₃ represents a hydrogen atom and R₄ represents a methyl group.

6. A method of preparing a compound according to any one of claims 1 to 5, characterised in that it comprises the steps consisting in:
1) allowing an amino acid of formula: 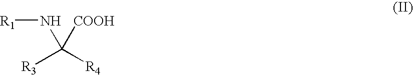
in which
R₁ represents an aromatic ring which is non-substituted or substituted with one or more atoms or groups of atoms selected from halogens, linear or branched C₁-C₄ alkoxy, linear, branched or cyclic C₁-C₄ alkyl, linear or branched C₁-C₄ alkylthio, nitro, trifluoromethyl, trifluoromethoxy, methylenedioxy or 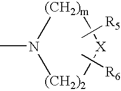
groups,
m represents 2 or 3,
X represents an oxygen atom, a sulphur atom, a sulphoxide group, a sulphonyl group, a carbonyl group, a 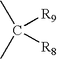
group, or a: 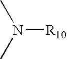
group,
R₃, R₄, R₅ and R₆ each independently represent a hydrogen atom or a C₁-C₄ alkyl group,
R₈ represents a hydrogen atom, a hydroxy group, a C₁-C₂ hydroxyalkyl group, a benzoyl group or a CO₂CH₃ group,
R₉ represents a hydrogen atom or forms, with R₈, an ethylenedioxy group,
R₁₀ represents a methyl group, a C₂-C₄ hydroxyalkyl group, a 1-oxo-C₂-C₄-alkyl group, an SO₂N(CH₃)₂ group, a 2-pyridinyl group or a 2-pyrimidinyl group,
to react with an isothiocyanate of formula
R₂—N═C═S (III)
in which R₂ represents
a linear, branched or cyclic C₁-C₇ alkyl group, optionally having one or more oxygen atoms,
a C₁-C₃ haloalkyl group,
a linear or branched C₃-C₅ alkenyl group,
a linear or branched C₃-C₄ alkynyl group,
a C₂-C₆ hydroxyalkyl group,
a protected C₂-C₄ aminoalkyl group,
a C₂-C₃ cyanoalkyl group,
a linear or branched C₁-C₃ alkyl group, which is optionally substituted with one or more R₇ substituents, or
an aromatic ring which is non-substituted or substituted with one or more atoms or groups of atoms selected from halogens, linear or branched C₁-C₄ alkoxy, linear, branched or cyclic C₁-C₄ alkyl, linear or branched C₁-C₄ alkylthio, cyano, hydroxy, nitro, trifluoromethyl, trifluoromethoxy, methylenedioxy, ethylenedioxy, difluoromethylenedioxy, aminosulphonyl, dimethylamino, C₁-C₃ hydroxyalkyl, carboxylic acid, C₂-C₃ alkyl ester, methanesulphonylamino, benzenesulphonylamino, t-butoxycarbonylamino, or 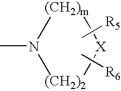
groups,
in a solvent, in the presence of an aprotic base, at a temperature of between 10° C. and the reflux temperature of the solvent, for 2 to 4 hours, to obtain the compound of formula I 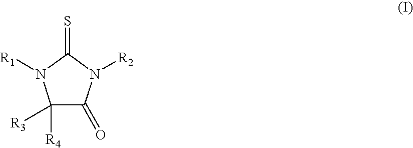
in which R₁, R₂, R₃, R₄ keep the same meaning as above, it being understood that at least one of the R₁ and R₂ groups contains in its structure an aromatic ring which is substituted at least by the 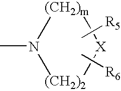
group, as defined above;
and,
2) if necessary, obtaining the addition salt of the compound of formula I above with an organic or mineral acid.

7. A method of preparing a compound according to any one of claims 1 to 5, characterised in that it comprises the steps consisting in
1) allowing an amino acid ester of formula (IIa) 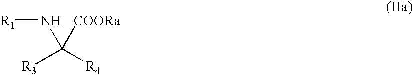
in which R₁, R₃ and R₄ have a meaning which is analogous to that of the R₁, R₃ and R₄ substituents which are noted for the compound of formula II which is described in the method A, and Ra represents a C₁-C₃ alkyl group, preferably an ethyl group,
to react with an isothiocyanate of formula
R₂—N═C═S (III)
as described above for the method A,
in a solvent, in the presence of a weak acid, at a temperature of between 50° C. and the boiling temperature of the solvent, for 2 to 25 hours, to obtain the compound of formula I 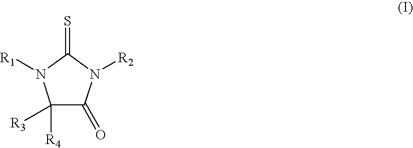
in which R₁, R₂, R₃, R₄ keep the same meaning as above, it being understood that at least one of the R₁ and R₂ groups contains in its structure an aromatic ring which is substituted at least by the 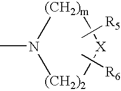
group, as defined above;
and,
2) if necessary, obtaining the addition salt of the compound of formula I above with an organic or mineral acid.

8. A method of preparing a compound according to any one of claims 1 to 5, characterised in that it comprises the steps consisting in
1) allowing an amino acid ester of formula (IIa) 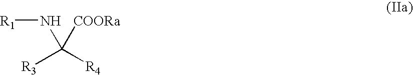
in which R₁, R₃ and R₄ have a meaning which is analogous to that of the R₁, R₃ and R₄ substituents which are noted for the compound of formula II which is described in the method A, and. Ra represents a C₁-C₃ alkyl group, preferably an ethyl group,
to react with an isothiocyanate of formula
R₂—N═C═S (III)
as described above for the method A,
in the presence of a weak acid, under microwave radiation, for 2 to 15 minutes, to obtain the compound of formula I 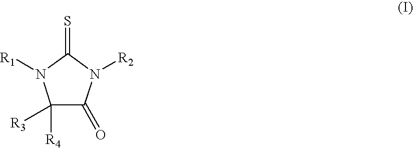
in which R₁, R₂, R₃, R₄ keep the same meaning as above, it being understood that at least one of the R₁ and R₂ groups contains in its structure an aromatic ring which is substituted at least by the 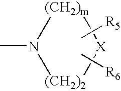
group, as defined above;
and,
2) if necessary, obtaining the addition salt of the compound of formula I above with an organic or mineral acid.

9. A pharmaceutical composition, characterised in that it contains, in combination with at least one physiologically acceptable excipient, at least one compound of formula I according to one of claims 1 to 5, or one of its addition salts with a pharmaceutically acceptable acid.

10. The compound of formula (I) according to any one of claims 1 to 5, or one of its addition salts with a pharmaceutically acceptable acid, for its use as a pharmacologically active substance.

11. Use of a compound of formula I according to one of claims 1 to 5, or one of its addition salts with a pharmaceutically acceptable acid, for the preparation of a medicament intended for treating diabetes or diseases caused by a hyperglycaemia.

12. Use of a compound of formula I according to one of claims 1 to 5, or one of its addition salts with a pharmaceutically acceptable acid, for the preparation of a medicament intended for treating hypertriglyceridaemiae and dyslipidaemiae.

13. Use of a compound of formula I according to one of claims 1 to 5, or one of its addition salts with a pharmaceutically acceptable acid, for the preparation of a medicament intended for treating obesity.

14. Use of a compound of formula I according to one of claims 1 to 5, or one of its addition salts with a pharmaceutically acceptable acid, for the preparation of a medicament intended for treating cerebral vascular accidents.
